(19) United States
(12) Patent Application Publication
(10) Pub. No.: US 2007/0224168 A1
(43) Pub. Date: Sep. 27, 2007

(54) Isolated Muscle Satellite Cells, Use Thereof in Muscle Tissue Repair and Method for Isolating Said Muscle Satellite Cells

(76) Inventors: Didier Montarras, Sevres (FR); Frederic Relaix, Paris (FR); Ana Simoes De Bivar Cumano, Paris (FR); Didier Rocancourt, Saint Martin De Nigelles (FR); Margaret Buckingham, Paris (FR)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 11/632,842

(22) PCT Filed: Jul. 19, 2005
(86) PCT No.: PCT/IB05/02561

§ 371 Date: Mar. 16, 2007

(30) Foreign Application Priority Data
Jul. 19, 2004 (CA) 2,475,174

(51) Int. Cl. A61K 35/34; A61P 21/00; C12N 5/06

(52) U.S. Cl. 424/93.1; 435/325; 435/7.2

(57) ABSTRACT

The present invention relates to the field of tissue engineering, and more particularly to isolated muscle satellite cells, their use for repairing damaged muscle tissues and a method for isolating said muscle satellite cells. Consequently, the present invention relates to isolated muscle satellite cells and a method for isolating muscle satellite cells, the use of such satellite cells in composition and method for repairing a damaged muscle tissue of a patient.

### FIELD OF THE INVENTION

The present invention relates to the field of tissue engineering, and more particularly to isolated muscle satellite cells, their use for repairing damaged muscle tissues and a method for isolating said muscle satellite cells.

### BACKGROUND OF THE INVENTION

Satellite cells are skeletal muscle progenitor cells responsible for post-natal growth and repair (Charge et al., 2004) for review. In adult muscles they are quiescent cells located between the muscle fibre plasmalemma and the surrounding basal lamina. The difficulty of isolating pure populations of satellite cells in sufficient number has precluded their use in cell based tissue repair assays. These assays have, therefore, employed muscle precursor cells that correspond to the progeny of muscle satellite cells, obtained after activation and proliferation in culture (Qu-Pertersen et al., 2002; Mueller et al., 2002; Skuk et al., 2002) or mixtures of cells obtained after enzymatic dissociation of skeletal muscles (Morgan et al., 1993; Morgan et al., 1996). In vivo, quiescent muscle satellite cells are characterized by the expression of surface markers such as M-cadherin, (Irintchev et al., 1994; Hollnagel et al., 2002), syndecan 3 and 4, (Cornelison et al., 2001) and CD34, (Beauchamp et al., 2000); none of which, however, permit unequivocal isolation because of lack of specificity or availability of suitable reagents. Satellite cells also express transcription factors, such as the myogenic determination factor, Myf5, (Beauchamp et al., 2000) and a member of the homeodomain/paired box family of Pax proteins, Pax7, (Seale et al., 2000).

Thus it will be apparent that such a muscle cell subset constitutes a target of choice in the field of muscle tissue repair. However, and according to the Applicant's knowledge, there is no method to this date for specifically isolating this type of muscle cells subset.

Therefore, there is a need for a method for isolating such muscle satellite cells.

### SUMMARY OF THE INVENTION

The present invention relates to a method that satisfies the above mentioned need.

More particularly, one object of the invention concerns a method for isolating muscle satellite cells, comprising the steps of:

a) providing a population of muscle cells; and

b) isolating from said population of muscle cells, muscle satellite cells having a low cellular granularity, a small size and bearing a CD34 marker.

Yet, the present invention has also for an object a composition for repairing damaged muscle tissue of a patient, comprising isolated muscle satellite cells having a low cellular granularity, a small size and bearing a CD34 marker.

Another object of the invention concerns a method for repairing a damaged muscle tissue of a patient, comprising the step of administering to said patient, an effective amount of the composition of the invention.

Other objects and advantages of the present invention will be apparent upon reading the following non-restrictive detailed description, made with reference to the accompanying figures.

### BRIEF DESCRIPTION OF THE FIGURES

FIG. 1. Pax3 expression in muscle satellite cells

A-C, Expression of Pax3 in different muscles from 3 week-old Pax3IRESnLacZ/+ mice, revealed by X-Gal staining. The Pax3 reporter is extensively expressed in diaphragm muscle (A), and in the gracilis, but not to the same extent in other hind limb muscles (B). Trunk muscles such as the serratus dorsali caudal contains many β-Galactosidase (β-Gal) positive cells, whereas the adjacent intercostales externi have very few labelled cells (C). g, gracilis; r, rib. D, Pax3 protein which has a molecular weight of 55 kDa is detected by western blot in protein extracts from different muscles (D: Diaphragm, L: Hindlimb, T: ventral Trunk muscles) in 3 week-old animals. Tubulin expression is shown as a loading control.

E-F′, Pax3IRESnLacZ/+ is expressed in a subset of diaphragm muscle nuclei from 3 week-old mice, as revealed by X-Gal staining (E, F) compared with DAPI staining (E′, F′) either in muscle transverse section (E-E′) or in isolated fiber (F-F′).

G-I′, β-Gal positive cells in the diaphragm muscle from 3 week-old Pax3IRESnLacZ/+mice are located in a muscle satellite cell position, as revealed by expression under the basal lamina marked with Laminin (G), or by co-expression with CD34 (H) or M-Cadherin (I). Corresponding DAPI staining is indicated (G′, H′, I′) for each panel. Arrows indicate the labelled satellite cell nuclei.

J-J′, Pax3 protein is shown using a Pax3-specific antibody, in the nucleus of a cell located under the basal lamina marked by a Laminin antibody (J). Corresponding DAPI staining is indicated, with the corresponding nucleus indicated by an arrow (J′).

K-M′, Co-immunohistochemistry on diaphragm muscle from 3 week-old Pax3IRESnLacZ/+ mice using antibodies recognizing Pax7 or β-Gal shows that in most cases Pax3 and Pax7 are co-expressed (K), however at low frequency one can find exclusive expression of Pax3 (L) or of Pax7 (M) on the same fiber. Corresponding DAPI staining is indicated and labelled nuclei are indicated by arrows (K′-M′) for each panel.

FIG. 2. Pax3 and Pax7 expression in satellite cell cultures.

A-B, Expression of Pax3 in primary cultures derived from the diaphragm of 3 week-old Pax3nLacZ/+ mice after 4 days (A) and 10 days (A′) of culture, visualized by X-Gal staining.

C, Histograms showing the number of β-gal positive colonies of myogenic cells obtained from diaphragm, trunk and hind limb muscles of 3 week-old Pax3mLacZ/+ mice. Cell were plated at low density, as described in methods to permit the formation of colonies and stained with X-Gal 3 to 4 days after plating. The results are from 3 independent experiments and after counting at least 100 colonies from cultures plated in triplicate.

D-I, Co-immunohistochemistry on primary cultures derived from the trunk muscles of 3 week-old Pax3mLacZ/+ mice using DAPI staining (D,G), or an antibody recognizing β-Gal (red, E,H) or MyoD (green, F) or Pax7 (green, I). Whereas β-Gal and MyoD are co-expressed in proliferating myoblasts, upon terminal differentiation Pax3 (β-Gal) is down-regulated (white arrow), and is already lower in some mononucleated MyoD positive cells (pink arrow).

J-O, Co-immunohistochemistry on primary cultures derived from the hind limb muscles of 3 week-old Pax3mLacZ/+ mice using DAPI staining (J,M), or an antibody recognizing β-Gal (red, K,N) or Pax7 (green, L,O). All cells are co-expressing β-Gal and Pax7. In limb muscles, colonies expressing either Pax7 alone (K,L) or Pax3 and Pax7 (N,O) were identified.

FIG. 3. Muscle satellite cells in newborn Pax7 mutant mice.

A-B′, Immunohistochemistry on transverse sections of ventral trunk muscle from Pax7LacZ/+ (A, A′) or Pax7LacZ/LacZ (B, B′) newborn (P2) mice using an antibody recognizing β-Gal (A′, B′). Corresponding DAPI staining is indicated (A-B). Arrowheads indicate nuclei expressing Pax7 (β-Gal).

C, Quantification of the number of β-Gal+ cells in Pax7LacZ/+ or Pax7LacZ/LacZ P2 mice, normalized to the number of fibers on 10 μm sections from ventral trunk muscle, showing a 20% reduction in the mutant mice at this stage.

D-G, Co-immunohistochemistry on transverse sections of ventral trunk muscle of Pax7LacZ/LacZ P2 mice using DAPI staining (D) or an antibody recognizing β-Gal (E) or M-Cadherin (F) shows that this satellite cell marker is co-expressed with Pax7 (G, arrowheads). M-cadherin is detectable on the surface of young fibers.

FIG. 4. Satellite cells in Pax7 mutant mice at P10.

A-D′, Co-immunohistochemistry on primary cultures derived from the diaphragm of Pax7LacZ/+ (A, A′, C, C′) or Pax7LacZ/LacZ (B, B′, D, D′) mice at P10 using DAPI staining (A, B, C, D) or antibodies recognizing Pax7 (A′, B′), MyoD (A′, B′, C′, D′) and Troponin T (C′, D′) shows the presence of myoblasts expressing MyoD and differentiated myotubes expressing MyoD and Troponin T.

E-F′, Co-immunohistochemistry on single fibers derived from the EDL muscle of Pax7LacZ/+ (E, E′) or Pax7LacZ/LacZ (F, F′) mice at P10, using antibodies recognizing M-cadherin (E, F) or CD34 (E′, F′).

G-H′, 68 hour cultures of single fibers derived from the EDL muscle of Pax7LacZ/+ (G, G′) or Pax7LacZ/LacZ (H, H′) mice at P10. Proliferating myogenic cells are always found in cultures of Pax7LacZ/+ single fibers (G, G′), whereas most (H) but not all (H′) single fibers derived from Pax7LacZ/LacZ mice contained myogenic cells.

FIG. 5. The number of satellite cells on muscle fibers isolated from Pax7 mutant mice at P10.

A, Determination of the number of CD34+/β-Gal+ cells per fiber on single fibers isolated from the EDL of Pax7LacZ/+ and Pax7LacZ/LacZ mice at P10 and examined immediately. Mean numbers and standard deviations are indicated. The number of satellite cells is reduced by 90% in Pax7 mutant mice at this stage.

B, Scoring of the number of DAPI+ myonuclei per fiber in single fiber preparations isolated from the EDL from Pax7LacZ/+ and Pax7LacZ/LacZ mice at P10. Mean numbers and standard deviations are indicated. The number of myonuclei per fiber is reduced by about 50% in Pax7 mutant mice.

C, Scoring of the number of mononucleated cells observed after 68 hours culture of single fiber preparations derived from the EDL of Pax7LacZ/+ or Pax7LacZ/LacZ mice at P10. The number of proliferating activated satellite cells is reduced by 90% in Pax7 mutant mice.

FIG. 6. Pax3 expression is maintained in Pax7 deficient satellite cells.

A, Western blot analysis of Pax3 expression in diaphragm or ventral trunk muscles isolated from Pax7LacZ/+ or Pax7LacZ/LacZ mice at P3. Tubulin (Tub) expression is shown as a loading control.

B, Co-immunohistochemistry on transverse sections of ventral trunk muscle of Pax7LacZ/LacZ mice at P2 using DAPI staining and antibodies which recognize Pax3. Laminin staining shows that the Pax3 positive cells in Pax7 mutant mice are present in a satellite cell position.

FIG. 7. The role of Pax3 and Pax7 in MyoD and Myf5 expression in satellite cells.

A-B, Co-immunohistochemistry on primary cultures from hind limb muscles of 3 week-old wild-type mice infected with adenoviral vectors encoding either GFP (Adeno-GFP) alone, GFP and a dominant negative (DN) form of Pax3 (Adeno GFP+Pax3DN) or GFP and a dominant negative form of Pax7 (Adeno GFP+Pax7DN), DAPI staining (A-B), or antibodies recognizing GFP (A-B), Myf5 (A) or MyoD (B) were employed. Whereas the expression of Pax3DN or Pax7DN had no effect on Myf5 expression (A), MyoD expression was inhibited under these conditions (B). Cells expressing lower levels of Pax-DN are indicated with a yellow arrowhead.

C, Similar experiments performed on primary cultures from 10 day-old Pax7 mutants indicate that a dominant negative form of Pax3 (Adeno GFP+Pax3DN) severely affects MyoD expression (white arrowheads), whereas the Adeno-GFP had no effect.

D, Quantitation of these results for satellite cell cultures infected with a dominant negative Pax3.

FIG. 8. Satellite cell survival in Pax7 mutant mice.

A, Co-immunohistochemistry on transverse sections of ventral trunk muscle of Pax7LacZ/+ or Pax7LacZ/LacZ newborn mice at P0 or P3 using DAPI staining or antibodies recognizing Desmin (red) or the activated form of Caspase3 (green). Apoptotic cells which are Desmin positive are present in muscles from Pax7 mutant mice (arrowheads).

B, Co-immunohistochemistry on transverse sections of ventral trunk muscle of Pax7LacZ/+mice at P2 using DAPI staining or antibodies recognizing Desmin (red) or β-Gal (green). Activated Pax7 (β-Gal) expressing satellite cells are Desmin positive (stars), whereas quiescent satellite cells are Desmin negative (arrowheads).

C-D, Co-immunohistochemistry on transverse sections of ventral trunk muscle of Pax7LacZ/LacZ mice at P2 (C) or P6 (D) using DAPI staining or antibodies recognizing the activated form of Caspase-3, Laminin (C) or β-Gal (D) antibodies show that the Pax7 mutant cells located in a satellite cell position are subject to apoptosis.

FIG. 9. Pax7 and Pax3 show divergent activities in activated satellite cells survival.

A, Infection of primary cultures from the hind limb muscles of wild type mice with the adenoviral vectors encoding GFP or the dominant negative forms of Pax3 (Pax3DN) or Pax7 (Pax7DN). Adenovirus infected cells which express GFP were selected by FACS cell sorting. Cell death in this cell population was assayed by Propidium Iodide (PI) staining of the cells. The percentage of dead cells (PI+ cells) was significantly increased in Pax7DN infected cells (71%), whereas it remained unchanged in Pax3DN infected cells (16%), compared to cells infected with Adenovirus (GFP) alone (29%).

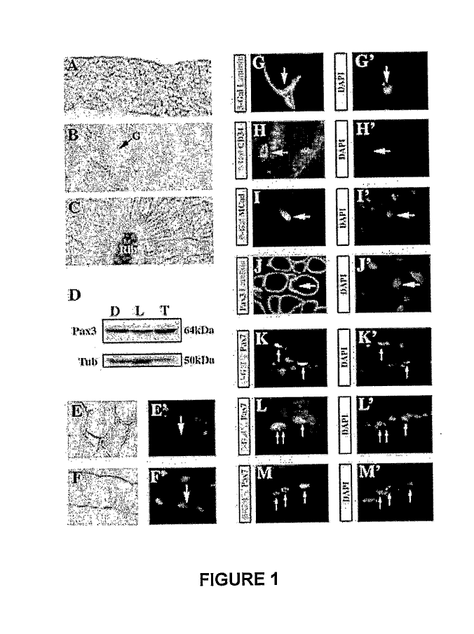

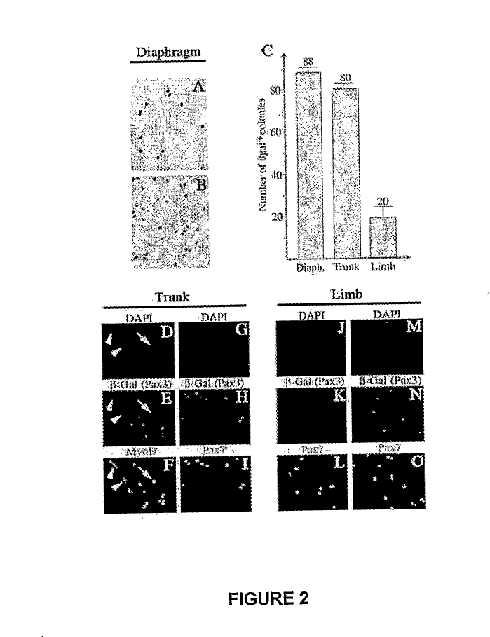

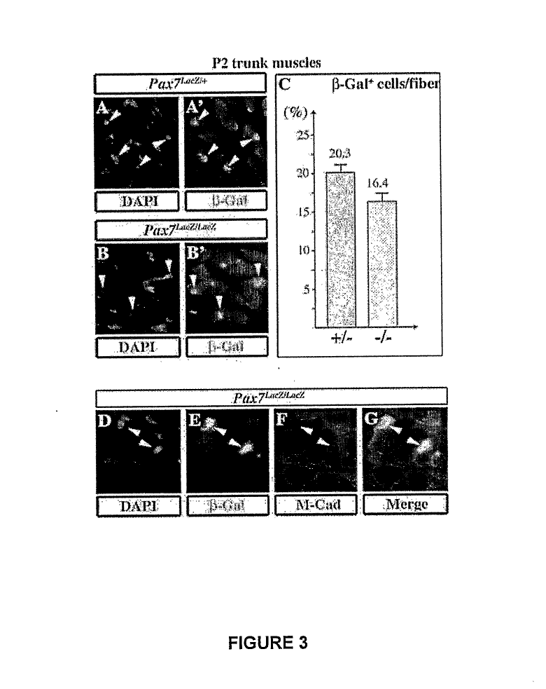

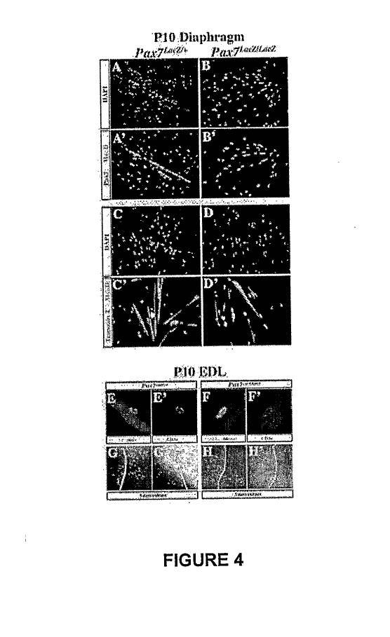

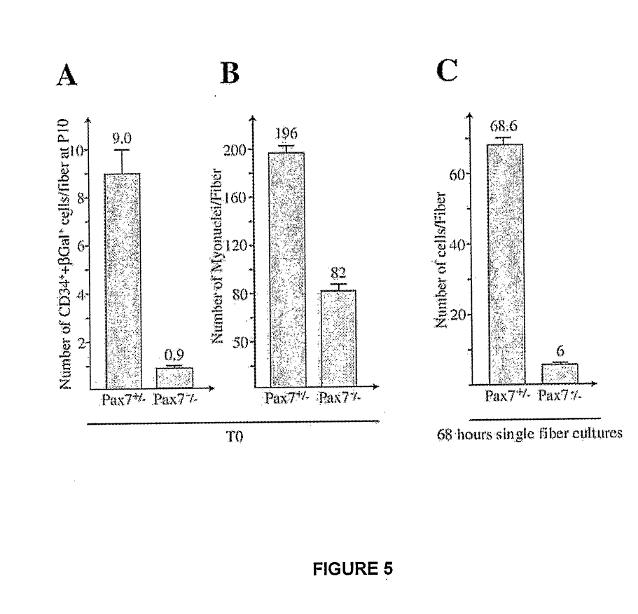

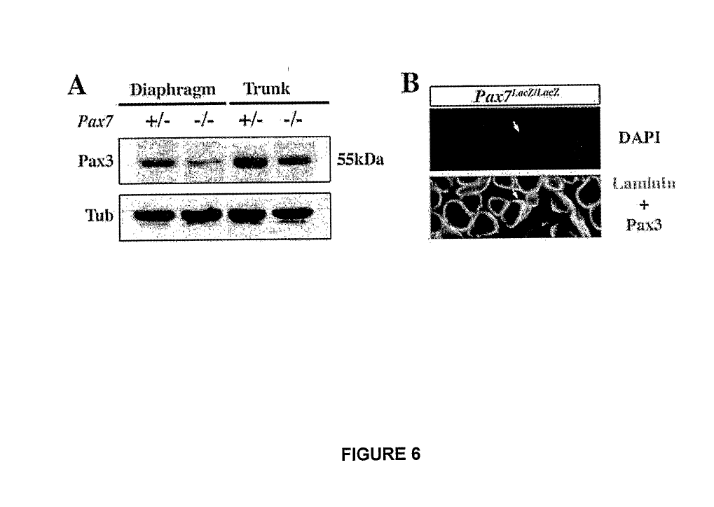

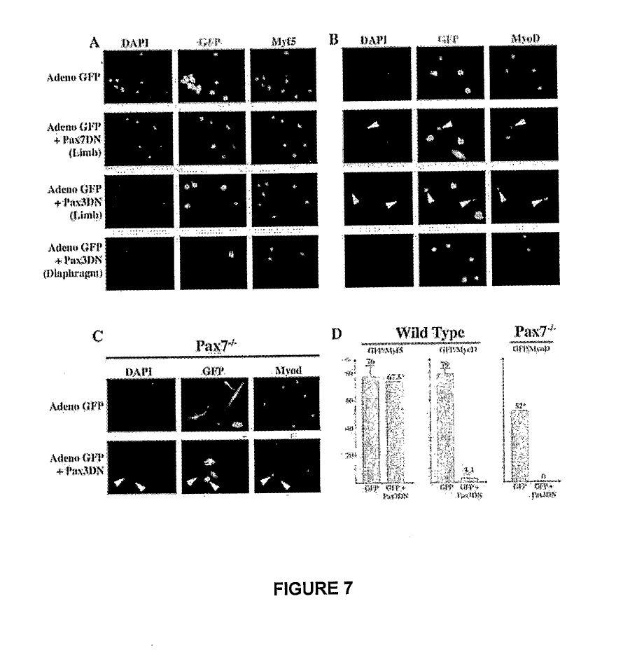

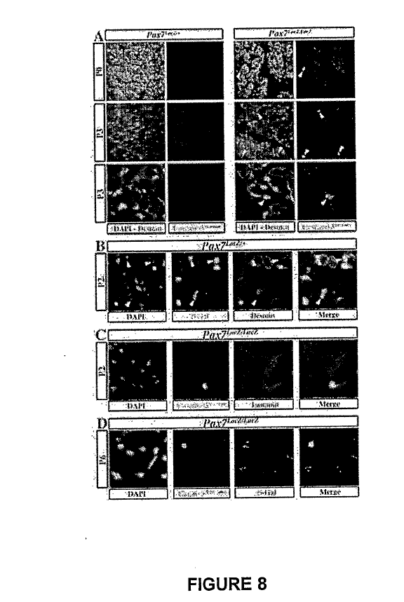

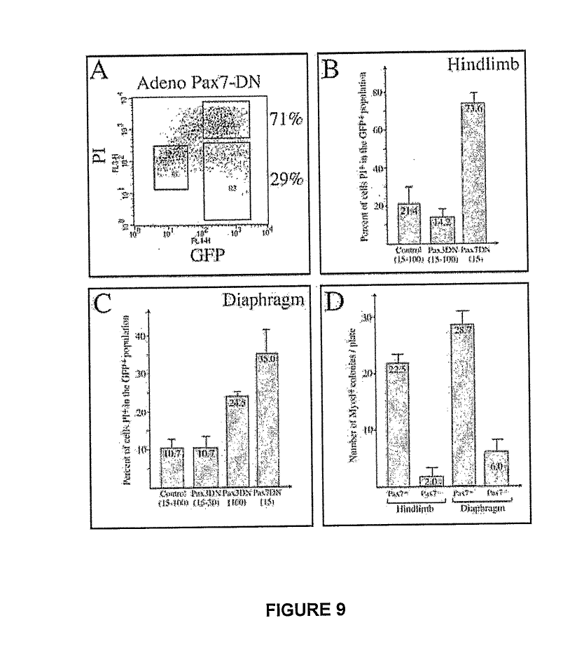

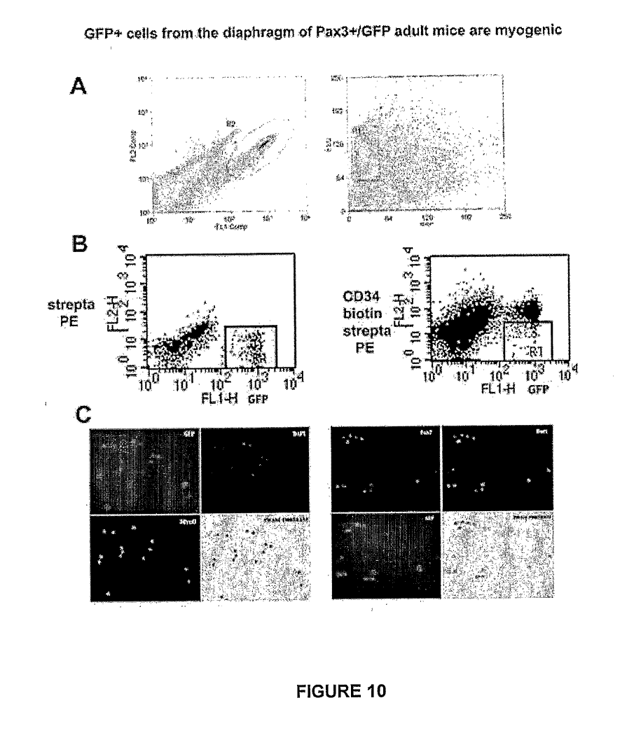

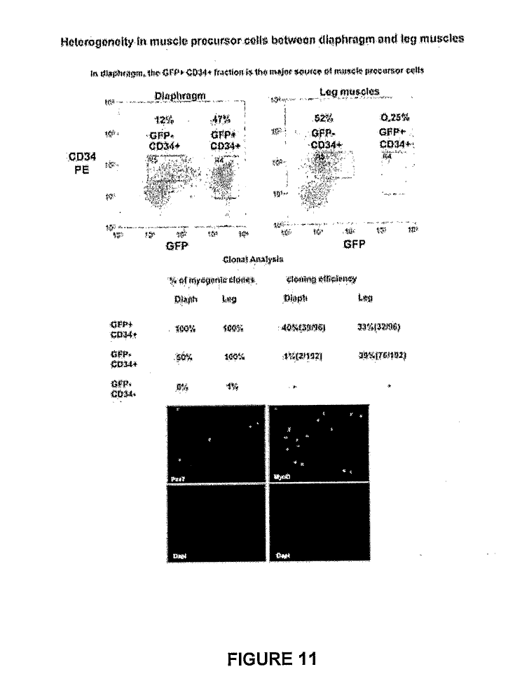

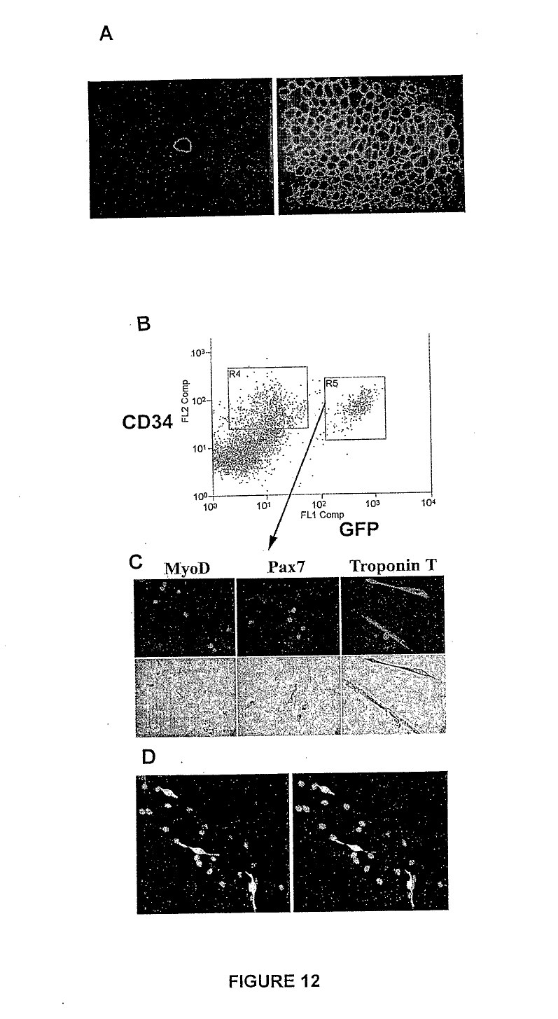

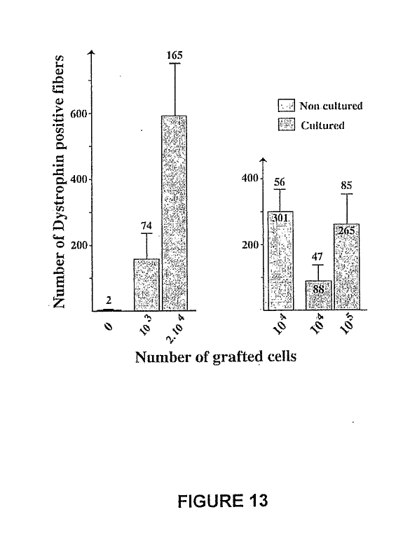

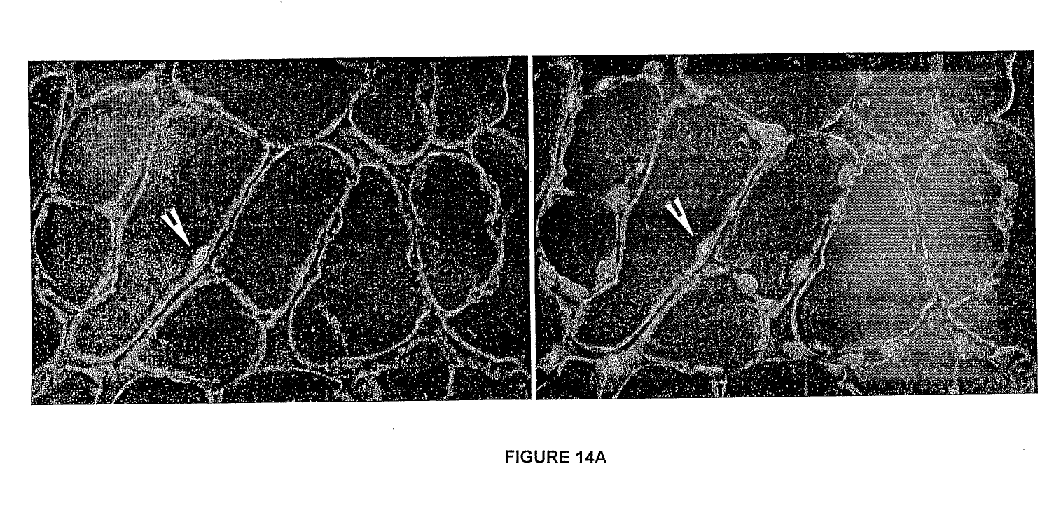

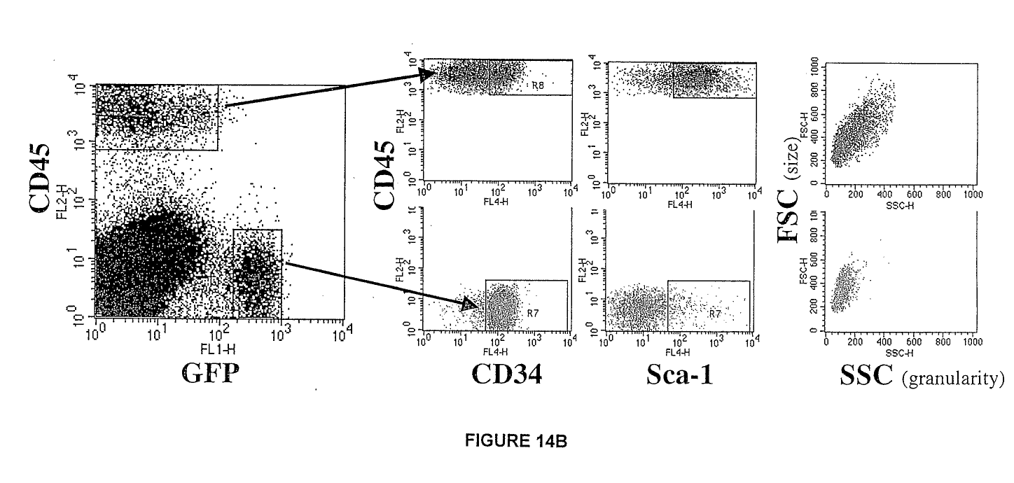

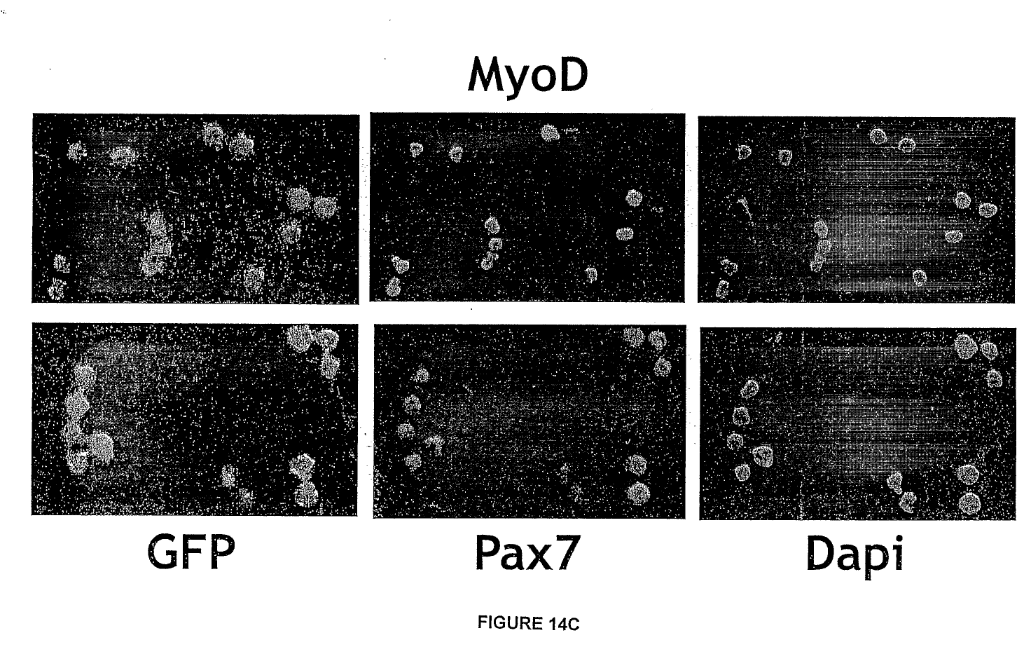

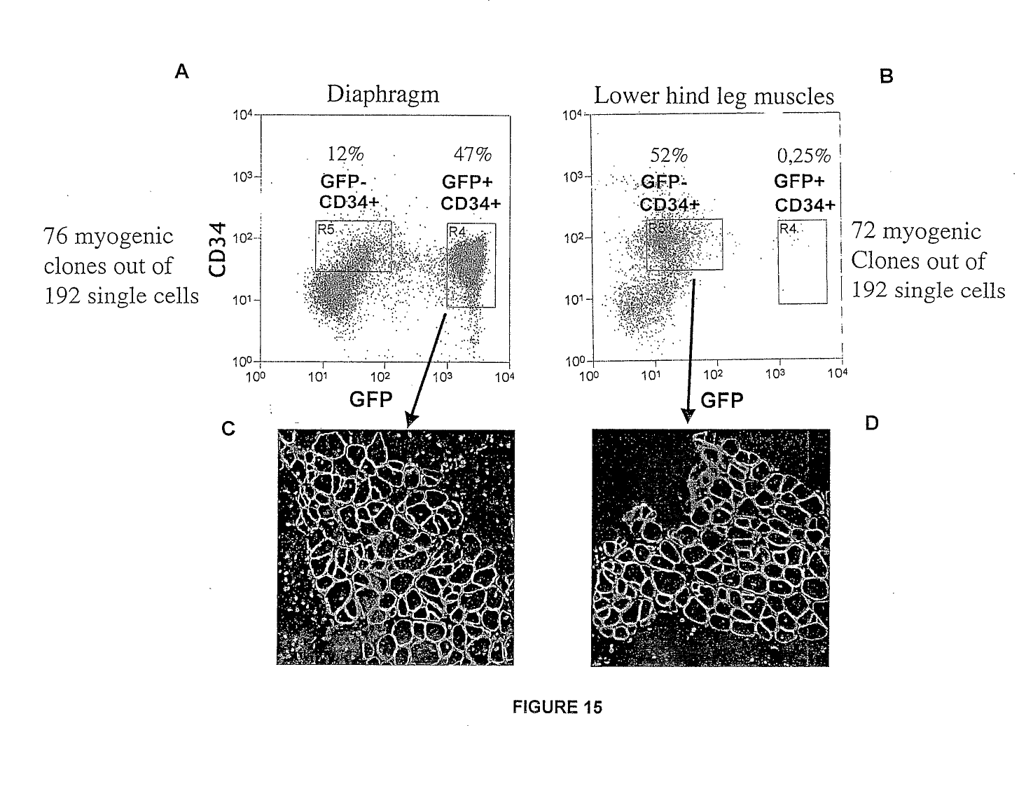

FIG. 10. Flow cytometry identifies a population of GFP+ events (window R2 FIG. 10A). Back gating of this R2 window to Forward Scatter (FSC) and Side Scatter (SSC) shows that the GFP+ events are confined into a window (R1) corresponding to cells of small size and low granulosity. FIG. 1B shows that the GFP positive cells isolated from the diaphragm are CD34+. FIG. 1C shows the myogenic identity (expression of MyoD and Pax7) of the (Pax3)GFP+ cells isolated by flow cytometry.

FIG. 11. Flow cytometry analysis of (Pax3)GFP+, CD34+ and (Pax3)GFP−, CD34+ cells from diaphragm and hind leg muscles. Flow cytometry and clonal analysis identify the GFP+CD34+ cell fraction as the major source of myosatellite cells in diaphragms whereas the major source of myosatellite cells of the hind leg muscles is found in the GFP-CD34+ fraction.

FIG. 12. FIG. 12A Dystrophin expression is restored in the GFP+ grafted cells. The fibers are red-colored with an antibody directed against dystrophin. Three weeks after grafting with (Pax3) GFP+ cells, TA muscles of mdx nu/lnu mice were processed for detection of dystrophin; right panel, tranverse section of grafted muscle; left panel, control contra-lateral non grafted TA. FIG. 12B Flow cytometry recovery of cells of donor origin (Pax3)GFP+ from grafted muscle. FIG. 12C Immediately after sorting, cells were plated and their myogenic identity determined by immunodetection of MyoD, Pax7 and Troponin T, after 3, 3 and 5 days of culture, respectively (upper panels). Lower panels show DAPI coloration and phase contrast. FIGS. 12B and 12C show that a subset of the grafted cells persists as mononucleated cells in the repaired muscle. These cells are myosatellite cells. FIG. 12D Detection of (Pax3)GFP+ muscle satellite cells on single fibres of grafted TA muscles. Single fibres were prepared from grafted muscles and processed for co-immunodetection of GFP and Pax7 as described in methods. Left panel, immunodetection of GFP with DAPI staining; right panel, co-immunodetection of Pax7 and GFP with DAPI staining on the same section. GFP marks both the cytoplasm and the nucleus of satellite cells, whereas Pax7 marks only the nucleus.

FIG. 13. Quantitative analysis of experiments as shown in FIG. 12A. Cells were grafted immediately after sorting (left panel). The effect of cell culture was examined by injecting cultured (C) or non-cultured (NC) cells (right panel). The numbers of injected mice were respectively from left to right 5, 4, 6, 5, 4.

FIG. 14. Characterization of (Pax3)GFP expressing cells. A) Transverse section of a diaphragm muscle from an adult Pax3^{GFP/+} mouse. Left panel, immunodetection of laminin (red staining), with DAPI coloration. Right panel, direct fluorescent detection of GFP+ cells (green staining) together with immunodetection of laminin. GFP positive cells are found in a satellite cell position in most muscles of adult Pax3^{GFP/+} adult mice. B) Flow cytometry analysis of the cells from the diaphragm of adult Pax3^{GFP/+} mice. CD34 and Sca1 expression on CD45+ cells, top panels and on GFP+ cells, bottom panels. Top and bottom right panels correspond to forward scatter (FSC) and side scatter (SSC) gating of CD45+ and GFP+ cells, respectively. C) Colony assay. (Pax3) GFP+ cells, isolated by flow cytometry from the diaphragm of Pax3^{GFP/+} mice, were plated at low density to permit colony formation and processed for immunodetection of MyoD and Pax7 three days later. 100% of the colonies (220 out of 220 n=2) were myogenic with respect to both markers. (Pax3)GFP+ cells constitute a homogeneous population of small non granular CD34+ CD45− Sca1-cells. IF analysis just after sorting shows that the (Pax3)GFP+ cell fraction consists of 93% of Pax7+ cells and 8% of MyoD+ cells.

FIG. 15. Isolation of muscle satellite cells in the absence of (Pax3)GFP expression. Flow cytometrics analysis of cells from diaphragm and lower hind leg muscles of adult Pax3^{+/GFP} mice. A) Cells from diaphragm muscles and B) from lower hind leg muscles, were analyzed for both GFP and CD34 expression as indicated in each panel. The percentages shown correspond to the fraction of positive cells within a FSC/SSC gate as shown in FIG. 14C. C) and D) Immuno-detection of dystrophin in TA muscles of mdx nu/nu mice 3 weeks after grafting 20 000 cells from the fractions indicated by the arrows. Both cell fractions contribute equally well to the restoration of dystrophin expression after grafting in TA muscles of mdx nude mice.

### DETAILED DESCRIPTION OF THE INVENTION

The present invention relates to the field of tissue engineering, and more particularly to isolated muscle satellite cells, their use for repairing damaged muscle tissues and a method for isolating said muscle satellite cells. Consequently, the present invention relates to isolated muscle satellite cells and a method for isolating muscle satellite cells, the use of such satellite cells in composition and method for repairing a damaged muscle tissue of a patient.

As used herein, the term “damaged muscle tissues” refers to a muscle tissue, such as a skeletal or cardiac muscle that has been altered for instance by an accident or a disease. A damaged muscle tissue according to a preferred embodiment may be a dystrophic muscle or an ageing muscle.

1. Method of Isolating

As a first embodiment, the present application provides a method for isolating muscle satellite cells, comprising the steps of:

a) providing a population of muscle cells; and

b) isolating from said population of muscle cells, muscle satellite cells having a low cellular granularity, a small size and bearing a CD34 marker.

It will be understood that the population of muscle cells are of animal origin and more preferably human origin.

It will be further understood that a low cellular granularity with regards to the satellite cells of the present invention may be determined by any method known to one skilled in the art, such as by density gradients determined for instance with Ficoll. However, the low cellular granularity according to a preferred embodiment of the invention is determined by flow cytometric analysis as a low side scatter (SSC) value. More preferably, the satellite cells have forward scatter (FSC) and SSC values as shown in gate R1 of FIG. 1A.

As it may be appreciated, step b) of the present method preferably consists of cell sorting and particularly achieved with a fluorescence activated cell sorter (FACS).

“Sorting” in the context of cells (e.g., “sorting a sample of muscle cells”) is used herein to refer to both physical sorting of the cells, as can be accomplished using, e.g., a fluorescence activated cell sorter (FACS), as well as to classifying (in the absence of physical separation) the cells based on expression of cell surface markers. The classifying may be done, for example, by simultaneously analyzing the expression of one or several markers, and determining the number and/or relative number of cells expressing different combinations of the markers (e.g., with the aid of a computer running a FACS analysis program).

“FACS” was originally coined as an acronym for Fluorescence Activated Cell Sorting, where the “Sorting” referred to physical separation of the cells into different containers. More recently, the use of term has broadened to include references to procedures and/or machines/instruments that relate to fluorescence analyses on a population of cells that result in a quantification of the number or relative number of cells having specific features, such as desired FSC and SSC values and/or selected levels of reporter fluorescence. The term “FACS” as used herein refers to the more recent, broader definition of the term.

According to a preferred embodiment and in order to make sure of the identity of the isolated cells as being muscle cells, the method of the invention preferably comprises an additional step of identifying a muscle specific transcription factor on said satellite cells obtained in step b). Preferably, the muscle specific transcription factor is MyoD. Identification of additional marker such as M-cadherin or syndecan-3 or -4 can be made.

It will be understood that the isolated muscle satellite cells are separated from the muscle tissue.

The isolating method of the present invention may further comprises another additional step of demonstrating myogenicity of said satellite cells obtained in step b). Such myogenicity of the cells is preferably determined by culturing the isolated muscle satellite cells of the invention in suitable conditions which are known by one of the art.

2. Method of Repairing and Compositions

In another embodiment, the present invention relates to a composition comprising isolated muscle satellite cells having a low cellular granularity, a small size and bearing a CD 34 marker.

Muscles satellite cells of the invention, may be used in many ways for repairing damaged muscle tissue.

In another embodiment, the present invention relates to a composition for repairing damaged muscle tissue of a patient, comprising a composition according to the invention, and an acceptable carrier.

In a preferred embodiment, said muscle satellite cells are obtained by the method according to the invention.

As used herein, the term “repairing” refers to a process by which the damages of a muscle tissue are alleviated or completely eliminated.

As used herein, the expression “an acceptable carrier” means a vehicle for containing the composition of the invention that can be administered into a host without adverse effects. Suitable carriers known in the art include, but are not limited to, liposomes, gold particles, sterile water, saline, glucose, dextrose, or buffered solutions. Carriers may include auxiliary agents including, but not limited to, diluents, stabilizers (i.e., sugars and amino acids), preservatives, wetting agents, emulsifying agents, pH buffering agents, viscosity enhancing additives, colors and the like.

Further agents can be added to the composition of the invention. For instance, the composition of the invention may also comprise agents such as drugs, immunostimulants (such as α-interferon, β-interferon, γ-interferon, granulocyte macrophage colony stimulator factor (GM-CSF), macrophage colony stimulator factor (M-CSF), interleukin 2 (IL2), interleukin 12 (IL12), and CpG oligonucleotides), antiapoptotic factors (such as insulin-like growth factors), antioxidants (such as ascorbic acid), surfactants, flavoring agents, volatile oils, buffering agents (such as buffer comprising a concentration of serum albumin close to the concentration of the animal serum), dispersants, propellants, and preservatives. For preparing such compositions, methods well known in the art may be used.

The amount of muscle satellite cells of the invention is preferably a therapeutically effective amount. A therapeutically effective amount of satellite cells of the invention is that amount necessary to allow the same to perform their myogenesis role without causing, overly negative effects in the host to which the composition is administered. The exact amount of satellite cells of the invention to be used and the composition to be administered will vary according to factors such as the type of muscle damage being repaired, the mode of administration, as well as the other ingredients in the composition.

The composition of the invention may be given to a host through various routes of administration. For instance, the composition may be administered in the form of sterile injectable preparations, such as sterile injectable aqueous or oleaginous suspensions. These suspensions may be formulated according to techniques known in the art using suitable dispersing or wetting agents and suspending agents. The sterile injectable preparations may also be sterile injectable solutions or suspensions in non-toxic parenterally-acceptable diluents or solvents. They may be given parenterally, for example intravenously, intramuscularly or sub-cutaneously by injection, by infusion or peros. It may also be administered into the airways of a subject by way of a pressurized aerosol dispenser, a nasal sprayer, a nebulizer, a metered dose inhaler, a dry powder inhaler, or a capsule. Suitable dosages will vary, depending upon factors such as the amount of each of the components in the composition, the desired effect (short or long term), the route of administration, the age and the weight of the host to be treated. Any other methods well known in the art may be used for administering the composition of the invention.

In a further embodiment, the present invention provides a method for repairing a damaged muscle tissue of a patient, comprising the step of administering to said patient, an effective amount of the composition as defined above. The step of administering the composition is preferably achieved by injecting the composition of the invention into and/or near the damaged muscle tissue.

As used herein, the term “patient” refers to a human or an animal.

### EXAMPLES

The present invention will be more readily understood by referring to the following examples. These examples are illustrative of the wide range of applicability of the present invention and are not intended to limit its scope. Modifications and variations can be made therein without departing from the spirit and scope of the invention. Although any methods and materials similar or equivalent to those described herein can be used in the practice for testing of the present invention, the preferred methods and materials are described.

The Pax3^{+/GFP} murine cell line has allowed the inventors to enhance the phenotypical and functional characterization of these cells. The Flow cytometry studies have shown that GFP myosatellite cells (Pax3) 1) constitute a cellular population of homogeneous size and morphology localized in a restricted frame determined by Forward Scatter and Side Scatter and 2) bear the surface marker CD34. The traits described in 1 and 2 allowed the inventors to isolate myosatellite cells from muscles that do not express the GFP (Pax3) gene. This observation is important because it allows a generalization of the procedure for isolating myosatellite cells from adult mouse muscle.

The inventors have determined that GFP (Pax3) cells effectively contribute to muscle repair in mice. In order to prove this, they have injected the GFP (Pax3) cells immediately following their isolation, by FACS, in dystrophic muscle of mdx mice which are dystrophin deficient. The results indicated that a small number of cells (a few thousands) is sufficient to restore dystrophin expression in many hundreds of fibres. These results are remarkable with regards to the number of cells (on the order of millions) usually injected by other researchers in the same animal model. It is also important to note that the cells injected by these researchers are not myosatellite cells, but cells derived from myosatellite cells following activation and amplification in culture. This is why the isolation method of the present invention is innovative since it allows for the isolation of myosatellite cells themselves.

In summary, the inventors have defined the conditions for isolating myosatellite cells based on GFP (Pax3) gene expression. This model has served as a guide to establish a general process for isolating myosatellite cells in order to use them for muscle cell therapy in mice. In this process, the CD34 surface marker is essential. Its presence on the surface of murine myosatellite cells has been evidenced by previous researchers (Zammmit et al., 2001), however the present results make it an instrument for the selection and the isolation of myosatellite cells for muscle repair.

The generation of a Pax3^{GFP/+} mouse line permitted to isolate (Pax3)GFP expressing cells from adult skeletal muscles by flow cytometry. Ex vivo and in vivo studies demonstrate that these cells constitute a pure population of muscle progenitors, expressing both Pax7 and CD34, that can contribute to muscle fibre repair and to the muscle satellite cell compartment where they retain Pax3 expression independently of their environment. These freshly isolated cells are markedly more efficient in tissue regeneration than their progeny obtained after activation and proliferation in culture. The instant invention provides evidence for muscle satellite cell self-renewal and paves the way for a general protocol applicable to the isolation of satellite cells and their therapeutic use for the repair of degenerated skeletal and cardiac muscle.

### Example 1

### Process for Obtaining a Preparation of Satellite Muscle Cells and Its Use for Skeletal Muscle Cell Therapy

After establishing that satellite muscle cells express the Pax3 gene (see also Example 3), the inventors undertook to isolate the muscle cells of the Pax3^{+/GFP} cell line based on the expression of the autofluorescent protein GFP (Green Fluorescent Protein) by FACS. The results from this analysis are presented in FIG. 10. After enzymatic dissociation of adult Pax3^{+/GFP} mouse diaphragms, the cellular suspension obtained was analyzed by FACS. A population of fluorescent cells was identified; it was localized in gate R2 (FIG. 10A). The analysis of these cells by Forward Scatter and Side Scatter, providing the inventors with information on the size and on the morphology of the cells, indicated that these fluorescent cells formed a homogeneous population of small sized cells localized in gate R1 (FIG. 10A).

The analysis of these cells after incubation with a CD34 surface protein antibody conjugated to biotin, and further incubation with avidin coupled phycoerythrin (FIG. 10B), indicated that the cells expressing GFP (abscissa axis) also express the CD34 protein. This finding is illustrated by the displacement of the cloud of points along the ordinate axis. The identity of the isolated cells as muscle cells was then established by underscoring the expression of specific transcription factors of the muscular lineage, such as MyoD factor (FIG. 10C) as well as noting the capacity of these cells to form muscular fibres after they have been cultured. All the clones formed by these cells were revealed to be myogenic. Taken together, these observations indicate that the isolated cells were indeed satellite muscle cells which, once activated, became precursor muscle cells which were able to proliferate and to differentiate into muscle fibres.

The process of isolating satellite muscle cells from muscles that express GFP protein under the control of Pax3 has served as a guide to isolate satellite muscle cells from muscles that do not express this marker gene. As a model, the inventors have used the hind leg muscles of Pax3^{+/GFP} mice, muscles which do not express GFP. The results from the FACS analysis are presented in FIG. 11. First, the results establish that the hind leg muscles are indeed lacking GFP-expressing cells in contrast to the cells in the diaphragm (gate R4). The use of CD34 surface marker and the frame representing the size and the morphology defined by diaphragm cells expressing GFP (Pax3) have allowed to isolate, from hind leg muscles, a population of small cells that express the CD34 protein (gate R5). Clonal analysis revealed that 100% of the clones formed by these CD34+GFP− cells are myogenic based on the expression of the MyoD gene and the capacity to form muscle fibres in culture. Also, these CD34+GFP− cells isolated in the hind leg have the same cloning efficacy as the CD34+GFP+ cells isolated from the diaphragm.

Taken together these results indicate that this process of cellular selection from murine Pax3 GFP muscles may be generalized, and as such, allows the isolation of satellite muscle cells from any animal muscle independently of GFP-marker expression placed under the control of the Pax3 gene.

Previous observations, on Pax3^{nlacz/+} mice indicated that satellite cells expressing the transcriptional regulator, Pax3, were limited to a subset of adult skeletal muscles, including diaphragm, most trunk muscles and some limb muscles. To introduce a fluorescent label into these cells, the Inventors generated a Pax3^{GFP/+} mouse line as described previously (Relaix et al., 2005). As illustrated in the diaphragm (FIG. 14A) (Pax3)GFP+ cells are found in a typical satellite cell position beneath the layer of laminin that surrounds muscle fibres. Most of these cells also express the transcriptional regulator, Pax7, which marks muscle satellite cells (Seale et al., 2000).

Flow cytometric analysis of the cells prepared from diaphragm muscle of adult Pax3^{GFP/+} mice (FIG. 14B) indicates that (Pax3)GFP+ cells constitute a population of CD45 negative cells, which are mainly Sca1 negative and CD34 positive. The majority, 95%, of the GFP+ cells in this experiment, were identified as CD34+ cells. This population is readily distinguishable from the blood cell population identified by the hematopoietic marker, CD45. Forward and side scatter gating (FIG. 14B left panel) indicated that (Pax3)GFP+ cells constitute a homogeneous population of small non-granular mononucleated cells. Immediately after sorting, immunodetection showed the presence of 93% Pax7+ cells and 8% MyoD+ cells. Pax7 marks both quiescent and activated satellite cells (Zammit et al., 2004), whereas MyoD marks activated satellite cells (Yablonka-Reuveni, 1994; Cornelison et al., 1997). This indicates that the majority of the cells have not undergone activation during the few hours (4 to 6 hours) that are required for dissociation and sorting. Colony assays further established the identity of these cells as muscle progenitors, giving rise to 100% of Pax7 and MyoD expressing cells after three days in culture (FIG. 14C). The (Pax3) GFP+ fraction that we have isolated thus constitutes a remarkably pure population of myogenic cells.

### Example 2

### Functionality of Satellite Muscle Cells Isolated by this Process

Restoration of Dystrophin Expression

The functionality of the satellite muscle cells that have been isolated by the method of the invention was evaluated in vivo following injection in the muscles of mdx, nude mice. The mice from the mdx line lack dystrophin, a protein of the mature muscular fibre. This mdx line was crossed with nude mice in order to attenuate the cell graft rejection phenomena. The results from the GFP+ cell grafts in the anterior tibialis muscle of these mice, immediately following their isolation by FACS, are presented in FIG. 12 and Table I.

TABLE I
Restoration of Dystrophin Expression

| | | Number of fibers |
|---|---|---|
| Number of cells injected | Number of mice | positive for dystrophin |
| 20 000 | 4 | 587 ± 165 |
| 1 000 | 5 | 160 ± 74 |
| 0 | 2 | 0 |

Six (6) cells are sufficient to restore the expression of dystrophin in a fibre.

These results indicate that a relatively small number of cells, a few thousands, is sufficient to restore dystrophin expression in many hundreds of fibres. To measure the efficiency of restoration of the cellular cultures, these results must be compared with those of other laboratories that do not obtain better results by injecting 100 to 1000 times more cells in the muscles of the same animal model.

Pax3)GFP+ cells, isolated by flow cytometry from adult diaphragm, were characterized functionally by grafting them into irradiated Tibialis Anterior (TA) muscles of immuno-deficient nude, mdx mice, (mdx nu/nu). These lack dystrophin, a structural protein that is mutated in Duchenne Muscular Dystrophy patients, (van Deutekom et al., 2003). Satellite cells of the TA, like those of other hindlimb muscles, do not normally express Pax3. The contribution of (Pax3)GFP+ cells to fibre repair was measured by the restoration of dystrophin expression in muscle fibres of host mice, 3 weeks after grafting. Numerous dystrophin+fibres were readily detected in the grafted muscles (FIG. 12A left panel). Only occasional dystrophin-positive fibres, probably revertant fibres, (Hoffman et al., 1990), were found in the control contra-lateral non-grafted TA (FIG. 12A, right panel). Grafting 2×10⁴ cells led to dystrophin expression in an average number of 587 fibres, while grafts of 10³ cells still resulted in dystrophin expression in an average of 160 fibres (FIG. 13 left panel). These yields are remarkable; comparable to those obtained after grafting 5×10⁵ cells isolated by enzymatic dissociation of whole adult muscles (Morgan et al., 1993; Morgan et al., 1996).

Most grafting experiments have employed muscle precursor cells obtained after a phase of amplification in culture (Qu-Petersen et al., 2002; Mueller et al., 2002). To determine whether such culturing procedures could alter the capacity of cells to contribute to tissue reconstitution, the Inventors grafted cultured and non-cultured (Pax3)GFP+ cells. Grafting 104 non-cultured cells led to restoration of dystrophin expression in an average number of 300 fibres, whereas, grafting the same number of cultured cells resulted in significantly fewer dystrophin-positive fibres (mean=88 p<0.02) (FIG. 13, right panel). The Inventors also grafted 10⁵ cells, corresponding to the progeny, after 3 days in culture, of 10⁴ flow cytometry isolated (Pax3)GFP+ cells. These cells led to restoration of dystrophin expression in an average number of 265 fibres, a figure that is similar to that obtained when grafting 10⁴ non-cultured cells (FIG. 13 right panel). These results show that culturing muscle satellite cells prior to grafting markedly reduces their efficiency in fibre repair, suggesting that in vivo expansion is not useful.

Characteristics of the Grafted Cells.

(Pax3) GFP+ CD34+ donor cells could be recovered by flow cytometry from grafted muscles (FIG. 12A). These cells displayed a myogenic phenotype in culture, expressing MyoD and Pax7, and differentiating into TroponinT expressing myotubes (FIG. 12B). Single fibres prepared from grafted muscles (FIG. 12C) carried cells of donor origin in a muscle satellite cell position, co-expressing (Pax3)GFP and Pax7. Of 569 cells detected on the surface of 120 single fibres from grafted TA muscles, 17% were satellite cells of donor origin co-expressing Pax7 and (Pax3)GFP. These results show that grafted muscle satellite cells not only contribute to muscle fibre repair, but also significantly to the muscle satellite cell compartment. They also show that (Pax3)GFP+ cells retain their Pax3+ identity in the environment of the TA muscle, where endogenous satellite cells do not express Pax3. Injured, as well as intact, TA muscle from Pax3^{GFP/+} mice does not normally contain (Pax3)GFP+ cells.

Flow cytometric analysis indicated that (Pax 3)GFP+ cells express the surface marker CD34. The Inventors used this surface marker and the parameters defined by forward scatter and side scatter gating for (Pax3)GFP+ cells (FIG. 14B) to determine whether muscle progenitor cells that do not express (Pax3)GFP could also be isolated from adult muscles. The GFP+CD34+ cells isolated from the diaphragm of adult mice (FIG. 15A) represented 47% of the cells analysed by flow cytometry. Clonal analysis of the cells from each fraction showed that of the clones formed, (78 out of 192 single cells) all were myogenic. In contrast, the GFP− CD34+ cells (FIG. 15A) displayed a cloning efficiency of 6% and gave rise to only 2 myogenic clones out of 192 plated cells. The same cell fractions from the lower hind leg muscles, (FIG. 15B), gave markedly different results. GFP+ CD34+ cells, representing only 0.25% of the cells, gave rise to clones (33 out of 96 single cells) all of which were myogenic. However, the GFP− CD34+ cells, which now represented 52% of the population, gave rise only to myogenic clones, with a cloning efficiency of 39% (76 out of 192 single cells). These results confirm that adult muscle progenitor cells belong to the (Pax3)GFP+ CD34+ cell fraction in the diaphragm, whereas, in lower hind leg muscles, they are in the (Pax3)GFP− CD34+ fraction. Thus, the parameters of size and granularity defined for (Pax3)GFP+ cells permit an equally efficient isolation of muscle satellite cells by sorting on the basis of CD34 expression. Skeletal muscle repair assays confirm and extend these observations. Grafting of GFP+ CD34+ cells from the diaphragm of adult mice or of GFP− CD34+ cells from lower hind leg muscles of the same mice, into TA muscles of mdx nu/lnu recipients produced comparable restoration of dystrophin expression (FIGS. 15C and 15D). Thus both preparations displayed similar capacity to participate in muscle fibre repair.

Comparative Study of Cultured (C) and Non-Cultured (NC) Cells, as Regards Cloning and Expansion.

The Inventors have also addressed the issue, of the influence of tissue culture, finding that the culture of muscle progenitor cells prior to grafting markedly reduces their regenerative efficiency. Indeed, the instant results show that the culture expansion itself is an ‘empty’ process, yielding the same amount of muscle as the number of cells from which the culture was intiated. Culture induced modifications may affect survival and/or engraftment capacity of the cells (DiMario et al., 1995). Indeed, the majority of cultured muscle precursor cells quickly die after grafting (Beauchamp et al., 1999). Equally, the activated state of the grafted cells may diminish their regenerative potential, since freshly isolated progenitor cells are not activated at the time of grafting, unlike their cultured progeny, which proliferate and express MyoD. A similar situation is encountered with hematopoietic stem cells, which lose their tissue reconstitution capacity when cultured (Antonchuk et al., 2002).

Moreover the expansion capacity in the context of the recipient has been compared between cultured cells and non-cultured cells according to the instant invention.

This study is performed by cloning cells in a 96 wells microplate.

Firstly, cells are sorted (Pax3+ CD34+), a first set of sorted cells are directly introduced in the wells of the microplate (1 cell/well; non-cultured cells [NC]); a second set of sorted cells are firstly cultured and secondly introduced in the other wells of said microplate (1 cell/well, cultured cells [C]).

Although the cloning efficiency is almost the same for the two sets of cells (NC and C) (about 50%) when the clones are counted in each well, the results show that a better expansion capacity is found in the context of the recipient with non-cultured cells (NC): the proliferation capacity is increased in non-cultured cells according to the invention.

These results are summed up in the following Table (Chi² test, P=10⁻²).

| Range of clones/well | Number in C | Number in NC |
|---|---|---|
| 1-20 | 17 | 1 |
| 21-50 | 23 | 10 |
| 51-100 | 12 | 20 |
| 101-200 | 8 | 14 |
| 201→ | — | 4 |

The better repair capacity of the NC cells must be dependent on their better proliferation potential.

Not only do purified non-cultureed muscle satellite cells contribute to muscle repair when engrafted into regenerating mdx muscles, but some persist as progenitor cells adopting a satellite cell position and expressing Pax7. These results, therefore, point to muscle satellite cell self-renewal. The fact that (Pax3)GFP+ cells can be recovered from the muscles into which they were originally transplanted and shown to differentiate into muscle cells in culture also argues in favour of self renewal. We, therefore, conclude that the satellite cell selection procedure described here results in cells, which can both repair and contribute to the progenitor cell population of damaged muscles. There may be other stem cell types that can be mobilized to contribute to this process (Partridge, 2004), for discussion, but the muscle satellite cell population isolated by the flow cytometry parameters that we have defined, is clearly a major contributor to muscle regeneration and a potential therapeutic agent.

### Example 3

### Pax7 is Required for Survival of Adult Muscle Satellite Cells, whereas Its Myogenic Function in Controlling MyoD is Shared with Pax3, Expressed in a Subset of Muscle

Pax7 and Pax3 share the capacity to control MyoD in adult muscle satellite cells whereas Pax7 is required for survival a function for which Pax3 does not compensate despite co-expression in a subset of muscles.

### Example 4

### Methods

Cell Culture

Cells were prepared from muscle tissue by enzymatic dissociation as previously described. Cells were plated on gelatin coated dishes in a 1:1 mixture (v/v) of F12 and DMEM medium (Gibco) containing 20% (v/v) FCS (AbCys), and 2% (v/v) ultroser (Biosepra). This medium that supports both proliferation and differentiation of muscle cells (Montarras et al., 2000) was used in all our experiments. To allow the formation of colonies of muscle cells, the plating density of primary cultures was comprised between 100 and 200 cells cm⁻², unless otherwise stated. Clonal analysis with fractions separated by flow cytometry (FIG. 4) was performed by automated plating of single sorted cells from each window shown in FIGS. 4A and 4B. Analysis of clones was performed 7 to 10 days after plating by immunodetection with MyoD and Pax7 antibodies and by detection of myotube formation.

Immunocytochemical Analysis.

Cells were treated as previously described (Montarras et al., 2000). Briefly, after fixation with 4% (w/v) PFA (paraformaldehyde) and permeabilization with 0.2% (w/v) Triton X100, cells were incubated with antibodies diluted in PBS containing 0.2% (w/v) gelatin. All incubations were at room temperature. For immunofluorescence, cells were mounted in mowiol (Calbiochem) after staining of DNA with bis-benzimide (DAPI) in the penultimate PBS wash.

Antibodies against MyoD were either a rabbit polyclonal antibody (Santa Cruz) used at 1:200 dilution or a mouse monoclonal antibody, clone 5.8A, (DAKO) used at 1:200 dilution. Antibodies against Pax7 and Troponin T were mouse monoclonal antibodies. Pax7: (Hybridoma Bank) used at 1:100 dilution. Troponin T: clone JLT 12 (Sigma) used at 1:200 dilution. Antibody against Laminin was a rabbit polyclonal antibody (L9393 Sigma) used at a 1:200 dilution. Antibody against GFP was a rabbit polyclonal antibody (Molecular Probe Inc) used at a 1:1000 dilution. Secondary antibodies were coupled to a fluorochrome, Alexa 488 or 594 (Molecular Probe, Inc).

Diaphragm and tibialis anterior muscles were removed and fixed in 4% PFA) and mounted in 7% gelatin and frozen under liquid nitrogen vapour. Cryosections of 12 microns were processed for immunodetection as described above.

Flow Cytometry

Flow cytometry analysis was preformed with an LSR analyzer (BD). Antibody against CD45 was a mouse monoclonal antibody clone 3° F.11, (BD) coupled to PE (phycoerythrin), antibodies against Sca1, clone D7, (BD) and CD34, clone RAM 34, (BD) were coupled to biotin and detected with streptavidin coupled to PE (phycoerythrin) or APC (Allophycocyanin). Cell sorting was performed with a Moflo (Cytomation).

Grafting of Donor Cells.

Three days prior to cell implantation, three week old mdx nu/nu (Partridge et al., 1989) host mice were anaesthetized with hypnorm and hypnovel (Gross et al., 1999). Both hind limbs were exposed to 18 Gy gamma radiation for 25 min (Gross et al., Cell Tissue Res, 1999). Irradiated mdx nu/nu mouse muscle provides an environment that encourages implanted muscle progenitor cells to proliferate (Beauchamp et al., 1999), to form new muscle and repair damaged host muscle (Morgan et al., 2002; Cousins et al., 2004) and to give rise to long-lived progenitor cells (Heslop et al., 2001; Morgan et al., 1994). Cell suspensions containing between 103 and 2×10⁴ cells in 4 μl of medium were prepared. Mice were anaesthetised with isofluorane and the skin overlaying the tibialis anterior (TA) muscle was opened. Cells were injected into the left and right TA muscles using a Hamilton 7005 syringe.

Analysis of Donor Muscle Formation.

TA muscles were removed for analysis three weeks after myoblast transplantation. Injected muscles were mounted in gum tragacanth (6% in water), frozen in liquid nitrogen-chilled isopentane and stored at −80° C. Cryosections from muscles implanted with each cell preparation were analyzed to assess the extent of donor cell contribution to repaired muscle fibres. 7 μm sections were cut from frozen embedded muscles on a Leica cryostat and immunostained with p7 rabbit polyclonal antibody against the carboxy terminal of dystrophin (Lu et al., 2005), detected by goat anti rabbit Alexa 594 (Molecular Probes). Slides were examined and photographed with a Zeiss Axiophot fluorescence microscope using Metamorph Imaging System (3.5) software. The number of donor (dystrophin-positive) muscle fibres was counted in representative transverse sections from the mid-belly of the injected muscle.

Contribution of Implanted Cells to the Satellite Cell Compartment.

TA muscles were removed three or four weeks after cell transplantation and viable muscle fibres isolated (Rosenblatt et al., 1995). These were fixed with 4% paraformaldehyde, permeabilized with 0.5% (WN) Triton X100 for 5 minutes and stained with antibodies for Pax 7 (Molecular Probes) and GFP (Molecular Probes). Pax 7 staining was visualised with goat anti-mouse Alexa 594 and GFP staining with goat anti-rabbit Alexa 488. The number of satellite cells expressing GFP and Pax7 or Pax7 alone was counted.

### Example 5

### Complementary Data

Introduction

Pax genes play key roles during development. Members of this family of homeodomain paired box transcription factors regulate the contribution of progenitor cells to different tissue types. During the formation of skeletal muscle in the embryo, Pax3 is an important player. The progenitor cells for most skeletal muscles are specified in the somites and this process depends on the myogenic regulatory proteins, basic-helix-loop-helix transcription factors which orchestrate both the determination of muscle cell fate and the differentiation of myoblasts into skeletal muscle fibres (Tajbakhsh and Buckingham, 2000). However in Pax3 mutant embryos skeletal muscles, such as those in the limbs, which form as a result of migration of myogenic progenitor cells from the somite, are absent (Bober et al., 1994; Franz et al., 1993; Goulding et al., 1994; Tremblay et al., 1998) and the hypaxial dermomyotome, the part of the dorsal somite from which such cells migrate, is missing. Furthermore Myf5/Pax3 double mutant mice lack all trunk as well as limb muscles, due to a failure in the activation of MyoD (Tajbakhsh et al., 1997), which, together with Myf5, acts as a myogenic determination gene. Recently it has been shown that another MyoD family member, Mrf4 was affected in the initial Myf5 mutant and that it can also act in muscle specification (Duchossoy et al., 2004). The replacement of a Pax3 allele by a PAX3-FKHR sequence, which as a fusion protein acts as a strong transcriptional activator, led to over-activation of Pax3 targets (Relaix et al., 2003). These include c-met required for muscle cell migration (Bladt et al., 1995) and MyoD, confirming that Pax3 lies genetically upstream of this myogenic regulatory gene. The PAX3-FKHR allele rescues the Pax3 mutant phenotype, showing that Pax3 acts as a transcriptional activator in the embryo.

A second Pax gene, Pax7, is also expressed in the somites and in myogenic cells in the embryo (Jostes et al., 1990). However it does not save the Pax3 mutant phenotype and indeed it is not expressed in the hypaxial dermomyotome or in migrating muscle progenitor cells in the mouse embryo (Relaix et al., 2004). Pax7 mutant embryos have no detectable muscle pheno-type (Mansouri et al., 1996), probaly because Pax3 is co-expressed in the subpopulation of Pax7 positive cells. In an experiment in which the Pax7 coding sequence was targeted into the Pax3 gene (Relaix et al., 2004), Pax7 was found to replace the function of Pax3 in the somites; the dermomyotome did not undergo apoptosis and trunk muscles formed normally. However the migration of muscle progenitor cells was affected and the formation of limb muscles was compromised, leading to the suggestion that after duplication of a common Pax3/Pax7 gene, present before vertebrate radiation, the functions of Pax3 and Pax7 diverged in response to the requirements of appendicular muscle formation.

Adult skeletal muscle undergoes regeneration when satellite cells, which lie under the basal lamina of muscle fibres, become activated, proliferate and form new skeletal muscle fibres, in response to damage (Bischoff and Heintz, 1994). Satellite cells also contribute to the postnatal growth of skeletal muscle. Myogenic regulatory genes are expressed during this process, Myf5 already in quiescent satellite cells (Beauchamp et al., 2000) and MyoD as they become activated and subsequently differentiate (Yablonka-Reuveni and Rivera, 1994). Myf5/MyoD double mutants have not yet been examined in this adult context because of the perinatal lethality of the original Myf5 mutants, however, in the absence of MyoD, muscle regeneration is less efficient and upon activation in culture, myosatellite cells display an abnormal phenotype (Megeney et al., 1996; (Only Megeney refers to in vivo, all the other authors, Yablonka 1999, Sabourin 2000, Cornelison 2000 and Montarras et al., 2000 have looked at primary cells from mutant mice). The striking result however came from examination of Pax7 mutant mice (Seale et al., 2000). In the absence of Pax7, satellite cells are absent from limb muscles and regeneration does not take place. Skeletal muscles are severely affected in adult Pax7−/− mice. These observations led to the proposal that Pax7 is essential for the specification of adult muscle progenitor cells, a function of the myogenic regulatory factors in the embryo (Seale et al., 2000). Thus, in the adult, Pax7, rather than Pax3, plays a predominant role. The presence of Pax3, however, has been documented in adult satellite cells after activation, leading to the proposal that it is implicated in their proliferation (Conboy and Rando, 2002b).

This example reports on the expression of Pax3 in the quiescent satellite cells of a subset of skeletal muscles, notably in those of the diaphragm and ventral body wall. The inventors show that both Pax3 and Pax7 control MyoD activation, as in the embryo. However their anti-apoptotic function differs. In the postnatal muscle of Pax7 mutant mice satellite cells are initially present and will differentiate in the presence of Pax3. In the absence of Pax7 these cells are progressively lost, indicating an essential anti-apoptotic role for Pax7 during postnatal myogenesis.

Results

Pax3 Expression in the Satellite Cells of Adult Skeletal Muscle

Analysis of adult mice in which the Pax3 gene is targeted with an nlacZ reporter (Relaix et al., 2003) revealed the presence of β-Galactosidase (β-Gal) positive cells in adult skeletal muscle. The number of such cells varies between muscles. They are particularly evident in the diaphragm (FIG. 1A), whereas they are much less frequent in hind limb muscles, with the exception of the gracilis muscle (FIG. 1B). Most ventral trunk muscles are positive, with a striking juxtaposition in the rib area, where intercostal muscles are mainly negative, whereas body wall muscles are positive (FIG. 1C). The Pax3 protein is also present as shown by western blot analysis of different muscles (FIG. 1D). Even in diaphragm muscle where there is extensive transcription of the nlacZ targeted Pax3 allele, only some nuclei are labelled (FIG. 1E,F). These correspond to satellite cells as shown by co-immunolocalisation of β-Gal with the satellite cell markers CD34 and M-Cadherin and by the inclusion of β-Gal positive cells within the basal lamina of the muscle fibre, labelled by a Laminin antibody (FIG. 1G-J). Since Pax7 is present in satellite cells (Seale et al., 2000), the question of Pax3 expression in relation to Pax7 in these cells was addressed. Although the majority of satellite cells are Pax7 positive, and Pax3 is co-expressed with Pax7, cells which express only Pax3 are also detected as shown for diaphragm muscle in FIG. 1K-M. We therefore conclude that Pax3, like Pax7, is expressed in quiescent satellite cells and that the frequency of this event varies between muscles, with no direct relation to fiber type since, for example, the mouse diaphragm contains mostly type I and IIX fibers which are labelled, whereas both the soleus (type I and IIA) and fast muscles such as the gastrocnemius (IIB) are mainly negative in the hind limbs.

When primary cultures are prepared from different muscles of Pax3nlacZ/+ mice, β-Gal positive cells are observed (FIG. 2A,B). The number of β-Gal positive colonies formed by satellite cells from different muscle sources was quantitated (FIG. 2C); the number of such colonies of activated satellite cells expressing Pax3nlacZ/+ in culture corresponds to the extent of β-Gal labelling of the different muscles in vivo. As the cultures begin to differentiate Pax3 expression is down-regulated in myotubes and already in some MyoD positive mono-nucleated cells (FIG. 2B, D-F). Co-expression with Pax7 is seen in cultures from muscles such as those of the trunk where Pax3 is also extensively expressed in satellite cells (FIG. 2G-I). However in cultures from the hind limb where this is less frequent, colonies of activated satellite cells which are only Pax7 positive are also found (FIG. 2J-L) as well as cells which co-express both Pax genes (FIG. 2M-O)

Satellite Cells and Muscle Fiber Formation in Pax7 Mutant Mice

Since Pax3 expressing satellite cells are found in adult muscles, their potential contribution to muscle growth and regeneration was investigated in the Pax7 mutant mouse. The inventors first examined muscles, such as those in the trunk, where Pax3 is extensively expressed in newborn Pax7lacZ/lacZ mice at postnatal (P) day two. Almost as many (80%) satellite cells, marked as β-Gal positive because they transcribe Pax7, were detected in mutant as in wild type mice at this stage (FIG. 3A,C). Co-expression with M-Cadherin confirmed that these are satellite cells (FIG. 3D-G).

At 10 days after birth satellite cells are still present in the diaphragm of Pax7 mutant mice, as shown in primary cultures in which MyoD positive cells are present and the cells form myotubes, with expression of differentiated markers (FIG. 4A-D′). As in the case of Pax3, Pax7 expression is down-regulated on differentiation, and indeed already in most MyoD positive myoblasts (FIG. 4A′). The EDL muscle from the forelimb still has occasional satellite cells marked with M-Cadherin or CD34 (FIG. 4E-F′) which are capable of proliferating when isolated fibers are cultured (FIG. 4G-H′). By this stage the number of satellite cells per fiber in the mutant is substantially reduced to about 10% (FIG. 5A). The overall number of nuclei (satellite cell and myonuclei) is also reduced by about half, indicating that muscle growth is also affected consistent with the role of satellite cells in this process (FIG. 5B). If Pax3 can compensate for Pax7, one might expect that satellite cells in muscles where Pax3 is extensively expressed would be less compromised at later stages, however this is not the case. For example when the same number of cells are isolated from ventral body wall or hind limb muscles of Pax7 mutant mice at P15, and cultured for 3 days, the number of MyoD positive cells in both cases is reduced to 5% of that seen with wild type mice under the same culture conditions. This indicates that there is a function(s) of Pax7 for which Pax3 cannot compensate.

The Mechanistic Role of Pax3 Relative to that of Pax7

When isolated fibers from Pax7 mutant mice at P10 are cultured for 68 hours, the number of activated satellite cells per fiber is reduced to about 10% of that seen when Pax7 is present (FIG. 5C). This figure is very similar to that observed for quiescent satellite cells in mutant versus normal mice (FIG. 5A). This indicates that satellite cell proliferation is not compromised in the absence of Pax7. It is important to be certain that Pax3 continues to be expressed in satellite cells in these mice. This is the case as shown in FIG. 6. Immunohistochemistry (FIG. 6B) and western blots show that Pax3 is still expressed, although at a reduced level (FIG. 6A), reflecting the progressive loss of satellite cells which is already more marked at postnatal day 3 (results not shown). In mouse embryos Pax3 plays a key role together with Myf5, in the activation of MyoD, such that in the absence of both Myf5 and Pax3, MyoD is not activated and the formation of skeletal muscle is compromised (Tajbakhsh et al., 1997). The inventors therefore investigated the relative roles of Pax3 and Pax7 in the activation of MyoD in adult satellite cells, using dominant negative constructs in which the Engrailed repression domain was fused to the —COOH terminal region of the Pax sequence, expressed in GFP marked adenovirus vectors. The results are shown in FIG. 7. The expression of dominant negative Pax3 and Pax7 constructs has no effect on Myf5 expression (FIG. 7A). However MyoD is absent or reduced in cells which express either of these vectors (FIG. 7B). This is observed in cultures from the limb, where satellite cells mainly express Pax7, and from the diaphragm, where most satellite cells are Pax3 and Pax7 positive. Since lower levels of the dominant negative Pax protein (yellow arrows, FIG. 7B) result in a lesser effect on MyoD levels in all cases, the inventors conclude that Pax3 and Pax7 have a similar affinity for the DNA targets which lead to this effect. In satellite cell cultures from Pax7 mutant mice MyoD is down regulated by expression of a dominant negative Pax3 (FIG. 7C). This confirms that Pax3 in this situation is responsible for MyoD activation. These results on the effects of the dominant negative Pax3 are presented quantitatively in FIG. 7D. The inventors therefore conclude that Pax3 as well as Pax7 can perform this function in satellite cells.

In order to try to explain the need for Pax7 in satellite cells which express Pax3 the inventors next investigated the survival of these cells in postnatal skeletal muscle. An antibody to the activated form of Caspase 3 was used as an indicator of apoptosis (Relaix et al., 2004). Muscles were labelled with an antibody to desmin which marks activated satellite cells as they assume a myoblast phenotype (Conboy and Rando, 2002a; Creuzet et al., 1998). In the postnatal skeletal muscle of Pax7 mutant mice Caspase 3 labelled cells are observed in contrast to control mice (FIG. 8A-D). These cells are also marked by the desmin antibody, suggesting that they correspond to activated satellite cells, probably contributing to the postnatal growth of muscle (FIG. 8A,B). The identification of these cells was confirmed by labelling with a laminin (FIG. 8C) or β-Gal antibody (FIG. 8D). The latter detects Pax7 transcripts in the mutant mice. In order to investigate the role of Pax3 compared to Pax7 in protecting against apoptosis, wild type satellite cells were transfected with GFP labelled adenovirus vectors expressing dominant negative Pax3 or Pax7. These cells were FACS sorted on the basis of GFP expression and their susceptibility to cell death was measured by Propidium Iodide staining which detects dying cells. It is clear from the results (FIG. 8A) that the dominant negative form of Pax7 leads to increased cell death in satellite cells (71%). Dominant negative Pax3, on the other hand, does not have this effect. This indicates that, unlike the situation for MyoD, it does not compete efficiently for targets of Pax7 which lead to protection from apoptosis in these satellite cells which were isolated from limb muscle. Since satellite cells in this muscle mainly contain Pax7 and not Pax3, we also carried out this experiment with satellite cells from diaphragm where Pax3 is widely expressed. In this case a high concentration of the dominant negative form of Pax3 also led to increased cell death (FIG. 8B . . . ), indicating that Pax3 can exert an anti-apoptopic effect on cells in which it is expressed. The anti-apoptotic effect of Pax3 is insufficient however to rescue satellite cells in Pax7 deficient mice in the longer term. The numbers of satellite cells isolated from the diaphragm, compared to limb muscle of Pax7 mutant mice is initially higher (FIG. 8C), but subsequently falls, consistent with the inventors observations at P15 that only 5% of satellite cells are present in either ventral body wall or hind limb muscles of mutant compared to wild type mice. Furthermore Caspase 3 positive cells are observed in diaphragm and trunk muscles where Pax3 is expressed (FIG. 9). The inventors therefore conclude that the major difference between Pax3 and Pax7 in postnatal satellite cells is their role as a survival factor. In Pax7 mutant mice, satellite cells are specified and are initially present. As they become activated during post-natal muscle growth they proliferate normally but they are progressively lost due to cell death. Pax3 cannot compensate for the cell survival function of Pax7.

Discussion

In the present analysis of the Pax7 mutant mouse the inventors show that satellite cells are initially present, indicating that these cells are specified in the absence of Pax7. Furthermore cell proliferation is not affected. Culture of cells from postnatal muscle indicates that the numbers of muscle cells immediately after birth (P1,2) are similar to wild type, but decline rapidly thereafter. While some of these cells, which express MyoD and form differentiated myotubes, may be a remnant of foetal myoblasts, the numbers of cells in the satellite cell position, expressing satellite cell markers, in mutant mice correlates with the results in culture, indicating that many of these are bona fide satellite cells. Satellite cells in Pax7 mutant mice undergo cell death after birth, visualised by the presence of large numbers of Caspase-3 positive cells on postnatal muscle sections. Caspase-3 positive cells are also clearly Desmin positive suggesting that they correspond to activated satellite cells (Conboy and Rando, 2002a; Creuzet et al., 1998). This would indicate that cell death intervenes during postnatal muscle growth. The anti-apoptotic effect of Pax7 is demonstrated by the death of cells isolated from skeletal muscle from the limbs of wild type mice when they are transfected with a dominant negative Pax7 protein.

It is probable that the specification of adult skeletal muscle cells depends on myogenic regulatory factors. Myf5 is expressed at a low level in satellite cells (Beauchamp et al., 2000) and it may be sufficient to determine myogenic identity. By analogy with embryonic myogenesis Pax7/Pax3 and/or Myf5 may perform this function, regulating MyoD transcription in activated satellite cells. Compound mutants for Pax7/Myf5/MyoD will clarify the adult gene hierarchy; this analysis is now accessible with the development of viable Myf5 mutants (Duchausoy et al., 2004; (Kaul et al., 2000). Transfection of satellite cell cultures with dominant negative Pax7 shows that MyoD but not Myf5 is down-regulated, consistent with a role for Pax7 in MyoD activation. In these experiments, surviving satellite cells are monitored, since the absence of Pax7 also leads to cell death. It is formally possible that only MyoD expressing cells are affected by apoptosis, however this is unlikely. While the effect on MyoD is detected immediately, cell death continues to increase over a longer period in culture. Contrary to what had been reported previously (Conboy and Rando, 2002a); not all activated satellite cells isolated from limb muscle express Pax3. As suggested by the experiment with dominant negative Pax7, Pax7 alone is sufficient for the expression of MyoD and subsequent differentiation. As MyoD begins to accumulate, Pax7 is down-regulated and is always absent from differentiating muscle cells.

The inventors show that Pax3 is expressed in quiescent satellite cells and that Pax7 is not unique in this respect. The introduction of an nlacZ reporter into an allele of Pax3 facilitated the appreciation of this phenomenon, which is also demonstrated at the protein level by western blotting and immunohistochemistry. Some of the Pax3 labelling is not in a satellite cell position and may correspond to cells in blood vessels and/or mesoangioblasts (De Angelis et al., 1999; Minasi et al., 2002), which transcribe the Pax3 gene (Buckingham, Cossu, unpublished observations). However the majority of Pax3 positive cells lie under the basal lamina of muscle fibres. Not all skeletal muscles have Pax3 positive satellite cells. Most hind limb muscles, such as the gastrocnemius which was the object of previous studies on the Pax7 mutant, are negative, whereas satellite cells in the proximal fore limb diaphragm and trunk (body wall) muscles express Pax3. There is no correlation with muscle fibre types. A link with the embryological origin of these muscles is also not evident. The diaphragm and ventral trunk muscles derive from the hypaxial dermomyotome, as do limb muscles. Furtheremore there is no evidence that the gracialis muscle which is positive for Pax3, is not formed by migrating progenitor cells like other muscles in the limb. The intercostal muscles, which are negative, probably form by elongation of the hypaxial dermomyotome as do body wall muscles which are positive. Heterogeneity between muscles is a well known feature of myopathies where a mutation in a gene expressed in all muscles has a pathological effect on particular muscle groups (Cao et al., 2003). It is also evident from the study of regulatory genes in the embryo that different sites of myogenesis are co-ordinated by different regulatory strategies. This is illustrated by the number of distinct sequences which control the spatio-temporal activation of the Myf5 gene (Buchberger et al., 2003; Hadchouel et al., 2003) or by the effects of mutations in genes encoding homeobox proteins such as Lbx1 (Brohmann et al., 2000; Gross et al., 2000; Schafer and Braun, 1999) or Mox2 (Mankoo et al., 1999) which lead to the loss of certain limb muscles and not others. Understanding the basis of heterogeneity between embryonic or adult muscles represents a challenge for the muscle field, which has tended not to think in these terms because of the apparently unilateral effects of the myogenic regulatory factors in the embryo.

In adult muscles in which Pax3 is present, Pax7 is co-expressed in most satellite cells, although all three categories—Pax3+, Pax3+/Pax7+ and Pax7+—are observed. Initially satellite cells which express Pax3 survive better. This is seen in the early postnatal period when cells are cultured from diaphragm compared to limb muscle. The anti-apoptotic effect of Pax3 in satellite cells is also shown by cell death observed on expression of a dominant negative form of Pax3. However the effect is distinct from that seen with a dominant negative Pax7. Firstly satellite cells from the limb, most of which do not express Pax3, are not affected. Secondly satellite cells from the diaphragm or body wall muscle where Pax3 is expressed, most frequently with Pax7, show a partial effect with either dominant negative Pax construct. These results therefore point to different targets for the anti-apoptotic effects of Pax3 and Pax7 in adult muscle. This is in contrast to the situation for MyoD which is a target of both Pax3 and Pax7. Although the presence of Pax3 initially protects satellite cells from cell death due to the absence of Pax7, in the longer term these cells also die, indicating that the cell death pathway normally blocked by Pax7 eventually dominates. In the embryo Pax3 is the factor which normally exerts an anti-apoptotic function in the hypaxial dermomyotome, and in its absence muscle progenitor cells from this part of the somite, which contribute to limb, diaphragm and trunk muscles, are lost. However when appropriately expressed, Pax7 can rescue this phenotype (Relaix et al., 2004). It is therefore possible that in the embryo these proteins have a common anti-apoptotic function, perhaps reflecting the role of the protein expressed in the somites of early vertebrates such as the cephalochordate amphioxus, which is encoded by a single Pax3/Pax7 gene (Holland et al., 1999). However, Pax7 rescue in the embryo may also be due to a distinct, but dominant antiapoptotic role for this Pax protein. In other tissue paradigms where Pax genes intervene, the emphasis has been on their role in cell fate choices, rather than cell survival. It is clear that during skeletal muscle formation, the antiapoptotic function of Pax3 and Pax7 is critical. In postnatal myogenesis, the presence of Pax7 in muscle satellite cells is essential for their survival.

The Inventors also show that flow cytometric analysis and characterization of (Pax3)GFP+ cells present in skeletal muscles of adult Pax3 GFP mice have permitted to define parameters for isolating adult muscle progenitor cells. These cells comprise a population of small, non-granular, CD34+ CD45− Sca1− cells expressing Pax7. Both Pax7 and CD34 had been shown previously to mark muscle satellite cells (Beauchamp et al., 2000; Seale et al., 2000). In accordance with this, a CD34+ cell fraction from skeletal muscles was shown to be enriched in myogenic cells (Jankowski et al., 2002). In a recent study, (Sherwood et al., 2004), adult muscle associated progenitor cells were also shown to belong to a fraction of CD45− Sca1− CD34+ cells. The purity of the preparations of adult muscle progenitor cells of the invention allows to demonstrate quantitatively their regenerative capacity, in vivo and to explore the expression of the two Pax genes.

Assays for muscle repair that have been developed, to date, are based on the injection of 5×10⁵ to, 10⁶ cells into the muscles of mdx mice. Most have been performed using cells, either directly obtained by enzymatic dissociation of muscles (Morgan et al., 1993; Morgan et al., 1996) or after a phase of selection and amplification in culture (Qu-Petersen et al., 2002; Mueller et al., 2002). When 5×10⁵ cells from freshly disaggregated muscle, were implanted into the TA of irradiated mdx nu/nu mice, they formed a mean of 328 dystrophin positive fibres (Morgan et al., 1993). Similar results were obtained after injecting one to two million muscle-derived cultured cells into limb muscle (Qu-Petersen et al., 2002; Mueller et al., 2002). The instant results now show that purified satellite cells are much more efficient than these crude/cultured cell populations in contributing to muscle repair.

Indeed the method according to the invention leads to NC cells having a significantly better capacity to proliferate compared to cultured (C) cells.

### REFERENCES

Antonchuk J., Sauvageau G., Humphries R. K., Ce/1109, 39-45 (Apr. 5, 2002).
Beauchamp J. R., Morgan J. E., Pagel C. N., Partridge T. A., J Cell Biol 144, 1113-22 (Mar. 22, 1999).
Beauchamp, J. R., Heslop, L., Yu, D. S., Tajbakhsh, S., Kelly, R. G., Wernig, A., Buckingham, M. E., Partridge, T. A., and Zammit, P. S. (2000). Expression of CD34 and Myf5 defines the majority of quiescent adult skeletal muscle satellite cells. J Cell Biol 151, 1221-1234.
Bischoff, R., and Heintz, C. (1994). Enhancement of skeletal muscle regeneration. Dev Dyn 201, 41-54.
Bladt, F., Riethmacher, D., lsenmann, S., Aguzzi, A., and Birchmeier, C. (1995). Essential role for the c-met receptor in the migration of myogenic precursor cells into the limb bud. Nature 376, 768-771.
Bober, E., Franz, T., Arnold, H. H., Gruss, P., and Tremblay, P. (1994). Pax-3 is required for the development of limb muscles: a possible role for the migration of dermomyotomal muscle progenitor cells. Development 120, 603-612.
Brohmann, H., Jagla, K., and Birchmeier, C. (2000). The role of Lbx1 in migration of muscle precursor cells. Development 127, 437-445.
Buchberger, A., Nomokonova, N., and Arnold, H. H. (2003). Myf5 expression in somites and limb buds of mouse embryos is controlled by two distinct distal enhancer activities. Development 130, 3297-3307.
Buckingham M. et al., J Anat 202, 59-68 (January, 2003).
Cao, B., Zheng, B., Jankowski, R. J., Kimura, S., Ikezawa, M., Deasy, B., Cummins, J., Epperly, M., Qu-Petersen, Z., and Huard, J. (2003). Muscle stem cells differentiate into haematopoietic lineages but retain myogenic potential. Nat Cell Biol 5, 640-646.
Charge S. B., Rudnicki M. A., Physiol Rev 84, 209738 (January, 2004).
Conboy, I. M., and Rando, T. A. (2002a). The regulation of notch signaling controls satellite cell activation and cell fate determination in postnatal myogenesis. Dev Cell 3, 397-409.
Conboy, I. M., and Rando, T. A. (2002b). The regulation of Notch signaling controls satellite cell activation and cell fate determination in postnatal myogenesis. Dev Cell 3, 397-409.
Cornelison D. D., Wold B. J., Dev Biol 191, 270-83 (Nov. 15, 1997).
Cornelison D. D., Filla M. S., Stanley H. M., Rapraeger A. C., Olwin B. B., Dev Biol 239, 79-94 (Nov. 1, 2001).
Cousins J. C., Woodward K. J., Gross J. G., Partridge T. A., Morgan J. E., J Cell Sci 117, 3259-69 (Jul. 1, 2004).
Creuzet, S., Lescaudron, L., Li, Z., and Fontaine-Perus, J. (1998). MyoD, myogenin, and desmin-nls-lacZ transgene emphasize the distinct patterns of satellite cell activation in growth and regeneration. Exp Cell Res 243, 241-253.
De Angelis, L., Berghella, L., Coletta, M., Lattanzi, L., Zanchi, M., Cusella-De Angelis, M. G., Ponzetto, C., and Cossu, G. (1999). Skeletal myogenic progenitors originating from embryonic dorsal aorta coexpress endothelial and myogenic markers and contribute to postnatal muscle growth and regeneration. J Cell Biol 147, 869-878.
DiMario J. X., Stockdale F. E., Exp Cell Res 216, 431-42 (February, 1995).
Franz, T., Kothary, R., Surani, M. A., Halata, Z., and Grim, M. (1993). The Splotch mutation interferes with muscle development in the limbs. Anat Embryol (Berl) 187, 153-160.
Goulding, M., Lumsden, A., and Paquette, A. J. (1994). Regulation of Pax-3 expression in the dermomyotome and its role in muscle development. Development 120, 957-971.
Gross J. G., Morgan J. E., Muscle Nerve 22, 174-85. (1999).
Gross J. G., Bou-Gharios G., Morgan J. E., Cell Tissue Res 298, 371-5 (November 1999).
Gross, M. K., Moran-Rivard, L., Velasquez, T., Nakatsu, M. N., Jagla, K., and Goulding, M. (2000). Lbx1 is required for muscle precursor migration along a lateral pathway into the limb. Development 127, 413-424.
Hadchouel, J., Carvajal, J. J., Daubas, P., Bajard, L., Chang, T., Rocancourt, D., Cox, D., Summerbell, D., Tajbakhsh, S., Rigby, P. W., and Buckingham, M. (2003). Analysis of a key regulatory region upstream of the Myf5 gene reveals multiple phases of myogenesis, orchestrated at each site by a combination of elements dispersed throughout the locus. Development 130, 3415-3426.
Heslop L. et al., Gene Ther 8, 778-83. (2001).
Hoffman E. P., Morgan J. E., Watkins S. C., Partridge T. A., J Neurol Sci 99, 9-25 (October, 1990).
Holland, L. Z., Schubert, M., Kozmik, Z., and Holland, N. D. (1999). AmphiPax3/7, an amphioxus paired box gene: insights into chordate myogenesis, neurogenesis, and the possible evolutionary precursor of definitive vertebrate neural crest. Evol Dev 1, 153-165.
Hollnagel A., Grund C., Franke W. W., Arnold H. H., Mol Cell Biol 22, 4760-70 (July, 2002).
Irintchev A., Zeschnigk M., Starzinski-Powitz A., Wernig A., Dev Dyn 199, 326-37 (April, 1994).
Jankowski R. J., Deasy B. M., Cao B., Gates C., Huard J., J Cell Sci 115, 4361-74 (Nov. 15, 2002).
Jostes, B., Walther, C., and Gruss, P. (1990). The murine paired box gene, Pax7, is expressed specifically during the development of the nervous and muscular system. Mech Dev 33, 27-37.
Kaul, A., Koster, M., Neuhaus, H., and Braun, T. (2000). Myf-5 revisited: loss of early myotome formation does not lead to a rib phenotype in homozygous Myf-5 mutant mice. Cell 102, 17-19.
Lu Q. L. et al., Proc Natl Acad Sci USA 102, 198-203 (Jan. 4, 2005).
Mankoo, B. S., Collins, N. S., Ashby, P., Grigorieva, E., Pevny, L. H., Candia, A., Wright, C. V., Rigby, P. W., and Pachnis, V. (1999). Mox2 is a component of the genetic hierarchy controlling limb muscle development. Nature 400, 69-73.
Mansouri, A., Stoykova, A., Torres, M., and Gruss, P. (1996). Dysgenesis of cephalic neural crest derivatives in Pax7−/− mutant mice. Development 122, 831-838.
Megeney, L. A., Kablar, B., Garrett, K., Anderson, J. E., and Rudnicki, M. A. (1996). MyoD is required for myogenic stem cell function in adult skeletal muscle. Genes Dev 10, 1173-1183.
Minasi, M. G., Riminucci, M., De Angelis, L., Borello, U., Berarducci, B., Innocenzi, A., Caprioli, A., Sirabella, D., Baiocchi, M., De Maria, R., et al. (2002). The meso-angioblast: a multipotent, self-renewing cell that originates from the dorsal aorta and differentiates into most mesodermal tissues. Development 129, 2773-2783.
Montarras, D., Lindon, C., Pinset, C., and Domeyne, P. (2000). Cultured myf5 null and myoD null muscle precursor cells display distinct growth defects. Biol Cell 92, 565-572.
Morgan J. E., Pagel C. N., Sherratt T., Partridge T. A., J Neurol Sci 115, 191-200 (April, 1993).
Morgan J. E. et al., Dev Biol 162, 486-98 (April, 1994).
Morgan J. E., Fletcher R. M., Partridge T. A., Muscle Nerve 19, 132-9 (February, 1996).
Morgan J. E. et al., J Cell Biol 157, 693-702 (May 13, 2002).
Mueller G. M., O'Day T., Watchko J. F., Ontell M., Hum Gene Ther13, 1081-90 (Jun. 10, 2002).
Partridge T. A., Morgan J. E., Coulton G. R., Hoffman E. P., Kunkel L. M., Nature 337, 176-9. (1989).
Partridge T., Cell 119, 447-8 (Nov. 12, 2004).
Qu-Petersen Z. et al., J Cell Biol 157, 851-64 (May 27, 2002).
Relaix, F., Polimeni, M., Rocancourt, D., Ponzetto, C., Schafer, B. W., and Buckingham, M. (2003). The transcriptional activator PAX3-FKHR rescues the defects of Pax3 mutant mice but induces a myogenic gain-of-function phenotype with ligand-independent activation of Met signaling in vivo. Genes Dev 17, 2950-2965.
Relaix, F., Rocancourt, D., Mansouri, A., and Buckingham, M. (2004). Divergent functions of murine Pax3 and Pax7 in limb muscle development. Genes Dev 18, 1088-1105.
Relaix F., Rocancourt D., Mansouri A., Buckingham M., Nature (Apr. 20, 2005).
Rosenblatt J. D., Lunt A. I., Parry D. J., Partridge T. A., In Vitro Cell Dev Biol Anim 31, 773-9. (1995).
Schafer, K., and Braun, T. (1999). Early specification of limb muscle precursor cells by the homeobox gene Lbx1h. Nat Genet 23, 213-216.
Seale, P., Sabourin, L. A., Girgis-Gabardo, A., Mansouri, A., Gruss, P., and Rudnicki, M. A. (2000). Pax7 is required for the specification of myogenic satellite cells. Cell 102, 777-786.
Sherwood R. I. et al., Cell 119, 543-54 (Nov. 12, 2004).
Skuk D., Goulet M., Roy B., Tremblay J. P., Exp Neurol 175, 112-26 (May, 2002).
Tajbakhsh, S., and Buckingham, M. (2000). The birth of muscle progenitor cells in the mouse: spatiotemporal considerations. Curr Top Dev Biol 48, 225-268.
Tajbakhsh, S., Rocancourt, D., Cossu, G., and Buckingham, M. (1997). Redefining the genetic hierarchies controlling skeletal myogenesis: Pax-3 and Myf-5 act upstream of MyoD. Cell 89, 127-138.
Tremblay, P., Dietrich, S., Mericskay, M., Schubert, F. R., Li, Z., and Paulin, D. (1998). A crucial role for Pax3 in the development of the hypaxial musculature and the long-range migration of muscle precursors. Dev Biol 203, 49-61.
van Deutekom J. C., van Ommen G. J., Nat Rev Genet 4, 774-83 (October, 2003).
Yablonka-Reuveni, Z., and Rivera, A. J. (1994). Temporal expression of regulatory and structural muscle proteins during myogenesis of satellite cells on isolated adult rat fibers. Dev Biol 164, 588-603.
Zammit P. S. et al., J Cell Biol 166, 347-57 (Aug. 2, 2004).

Although preferred embodiments of the present invention have been described in detail herein and illustrated in the accompanying drawings, it is to be understood that the invention is not limited to these precise embodiments and that various changes and modifications may be effected therein without departing from the scope or spirit of the present invention.

1. Method for isolating muscle satellite cells, comprising the steps of:
a) providing a population of muscle cells; and
b) isolating from said population of muscle cells, muscle satellite cells having a low cellular granularity, a small size and bearing a CD34 marker.

2. The method according to claim 1, wherein said low cellular granularity is determined by flow cytometric analysis as a low side scatter (SSC) value.

3. The method according to claim 1, wherein the satellite cells have forward scatter (PSC) and SSC values as shown in gate R1 of FIG. 1A.

4. The method of claim 1, wherein step b) comprises cell sorting.

5. The method of claim 4, wherein said cell sorting is achieved with a fluorescence activated cell sorter (FACS).

6. The method of claim 1, further comprising a step of identifying a muscle specific transcription factor on said satellite cells obtained in step b).

7. The method of claim 6, wherein the muscle specific transcription factor is MyoD.

8. The method of claim 1, further comprising a step of demonstrating myogenicity of said satellite cells obtained in step b).

9. A composition comprising isolated muscle satellite cells having a low cellular granularity, a small size and bearing a CD34 marker.

10. The composition of claim 9, wherein said muscle satellite cells are obtained by a method comprising the steps of:
a) providing a population of muscle cells; and
b) isolating from said population of muscle cells, muscle satellite cells having a low cellular granularity, a small size and bearing a CD34 marker.

11. Composition for repairing damaged muscle tissue of a patient, comprising the composition according to claim 9, and an acceptable carrier.

12. The composition of claim 11, wherein the muscle satellite cells are obtained by a method comprising:
a) providing a population of muscle cells; and
b) isolating from said population of muscle cells, muscle satellite cells having a low cellular granularity, a small size and bearing a CD34 marker.

13. Method for repairing a damaged muscle tissue of a patient, comprising the step of administering to said patient, an effective amount of the composition as defined in claim 11.

14. The method according to claim 13, wherein said step of administering the composition is achieved by injecting said composition into and/or near the damaged muscle tissue.

15. The method according to claim 13, wherein the damaged muscle tissue is a tissue chosen from a skeletal muscle tissue or a cardiac muscle tissue.

16. The method according to claim 13, wherein the damaged muscle tissue consists comprises of a dystrophic muscle or an ageing muscle.
